(19) United States
(12) Patent Application Publication
(10) Pub. No.: US 2011/0280941 A1
(43) Pub. Date: Nov. 17, 2011

(54) COMPOSITIONS OF PROTON PUMP INHIBITORS AND INSULIN SECRETAGOGUES, AND METHODS OF THEIR USE TO TREAT DIABETES

(75) Inventor: Ivan Newton Mefford, Sugarland, TX (US)

(73) Assignee: Mefford Scientific, LLC, Sugarland, TX (US)

(21) Appl. No.: 13/139,124

(22) PCT Filed: Dec. 11, 2009
(86) PCT No.: PCT/US2009/067733

§ 371 Date: Jun. 10, 2011

Related U.S. Application Data
(60) Provisional application No. 61/121,760, filed on Dec. 11, 2008.
(60) Provisional application No. 61/121,748, filed on Dec. 11, 2008.

(51) Int. Cl. A61K 9/36; A61K 31/444; A61P 3/10; A61K 31/451; A61K 9/28; A61K 31/4439; A61K 31/472

(52) U.S. Cl. 424/475; 514/338; 514/303; 514/309; 514/331; 424/474

(57) ABSTRACT

The present invention relates to compositions, kits and methods of treating diabetes. More specifically, compositions of at least one proton pump inhibitor and at least one insulin secretagogue, kits and methods of their use to treat diabetes.

This application claims the benefit of priority, under 35 U.S.C. 119(e), to U.S. Provisional Application Ser. No. 61/121,748, filed Dec. 11, 2008, titled “ORAL METHOD FOR TREATING DIABETES WITH A PHARMACEUTICAL COMPOSITION,” to U.S. Provisional Application Ser. No. 61/121,760, filed Dec. 11, 2008, titled “COMPOSITION FOR TREATING DIABETES,” and to PCT/US2009/067733, filed Dec. 11, 2009, titled “COMPOSITIONS OF PROTON PUMP INHIBITORS AND INSULIN SECRETAGOGUES, KITS AND METHODS OF THEIR USE TO TREAT DIABETES,” which are both herein incorporated by reference.

### BACKGROUND OF THE INVENTION

1. Field of the Invention

The present invention relates to compositions, kits and methods of treating diabetes. More specifically, compositions of at least one proton pump inhibitor and at least one insulin secretagogue, kits and methods of their use to treat diabetes.

2. Description of the Related Art

Diabetes mellitus is a disorder in which the pancreas cannot create sufficient insulin (Type I or insulin dependent) and/or in which insulin is not effective (Type 2 or non-insulin dependent). In the diabetic state, the victim suffers from high blood sugar, which may cause an array of physiological derangements (for example, kidney failure, skin ulcers, or bleeding into the vitreous of the eye) associated with the deterioration of small blood vessels. A hypoglycemic reaction (low blood sugar) may be induced by an inadvertent overdose of insulin, or after a normal dose of insulin or glucose-lowering agent accompanied by extraordinary exercise or insufficient food intake.

A need exists for a composition for the treatment of diabetes to reduce hemoglobin A1c in a mammal. With 24 million diabetics in the United States and approximately 300 million diabetics worldwide as of 2008, a need exists for a treatment of diabetes.

### SUMMARY OF THE INVENTION

In one embodiment, the invention relates to a kit having a container containing at least one proton pump inhibitor and at least one insulin secretagogue; and instructions for using the at least one proton pump inhibitor and the at least one insulin secretagogue for the treatment of diabetes in a method. The method is identifying a mammal suspected of having diabetes, and administering an effective amount of the one proton pump inhibitor and the at least one insulin secretagogue to the mammal.

In another embodiment, the present invention provides a composition to treat diabetes in a mammal having diabetes, the composition comprising a therapeutically effective amount of at least one proton pump inhibitor and a therapeutically effective amount of at least one insulin secretagogue. In another embodiment, the amount of the at least one proton pump inhibitor and the amount of the at least one insulin secretagogue will reduce hemoglobin A1c in the mammal by at least 1.5%. In another embodiment, the at least one proton pump inhibitor and the at least one insulin secretagogue are administered to the mammal daily for a period of at least 6 weeks and up to 24 weeks. In another embodiment the at least one proton pump inhibitor and the at least one insulin secretagogue are administered to the mammal daily for a period of at least 6 weeks. In another embodiment, the at least one proton pump inhibitor and the at least one insulin secretagogue are administered to the mammal daily for a period of at least 24 weeks. In another embodiment, the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, periprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof. In another embodiment, the at least one proton pump inhibitor is given to the mammal in a daily dose of at least 10 mg and the at least one insulin secretagogue is give to the mammal in a daily dose of at least 0.1 mg. In another embodiment, the at least one proton pump inhibitor is given to the mammal in a daily range of at least 5 mg and at most 1,000 mg and the at least one insulin secretagogue is give to the mammal in a daily range of at least 0.1 mg and at most 500 mg. In another embodiment, the composition is in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

In another embodiment, the present invention provides a composition to treat diabetes in a mammal having diabetes, the composition having a therapeutically effective amount of at least one proton pump inhibitor and a therapeutically effective amount of at least one insulin secretagogue so that the at least one proton pump inhibitor and the at least one insulin secretagogue when administered to the mammal cause an increase of the mammal's gastrin concentration levels compared to the mammal's baseline, the gastrin concentration levels increase thereof leading to a reduction of the mammal's hemoglobin A1C. In another embodiment, the amount of the at least one proton pump inhibitor and the amount of the at least one insulin secretagogue will reduce hemoglobin A1c in the mammal by at least 1.5%. In another embodiment, the at least one proton pump inhibitor and the at least one insulin secretagogue are administered to the mammal daily for a period of at least 6 weeks and up to 24 weeks. In another embodiment the at least one proton pump inhibitor and the at least one insulin secretagogue are administered to the mammal daily for a period of at least 6 weeks. In another embodiment, the at least one proton pump inhibitor and the at least one insulin secretagogue are administered to the mammal daily for a period of at least 24 weeks. In another embodiment, the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, periprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof. In another embodiment, the at least one proton pump inhibitor is given to the mammal in a daily dose of at least 10 mg and the at least one insulin secretagogue is give to the mammal in a daily dose of at least 0.1 mg. In another embodiment, the at least one proton pump inhibitor is given to the mammal in a daily range of at least 5 mg and at most 1,000 mg and the at least one insulin secretagogue is give to the mammal in a daily range of at least 0.1 mg and at most 500 mg. In another embodiment, the composition is in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

In another embodiment, the present invention provides a composition for reducing hemoglobin A1C in a mammal, the composition having a therapeutically effective amount of at least one proton pump inhibitor and a therapeutically effective amount of at least one insulin secretagogue. In another embodiment, the amount of the at least one proton pump inhibitor and the amount of the at least one insulin secretagogue will reduce hemoglobin A1c in the mammal by at least 1.5%. In another embodiment, the at least one proton pump inhibitor and the at least one insulin secretagogue are administered to the mammal daily for a period of at least 6 weeks and up to 24 weeks. In another embodiment the at least one proton pump inhibitor and the at least one insulin secretagogue are administered to the mammal daily for a period of at least 6 weeks. In another embodiment, the at least one proton pump inhibitor and the at least one insulin secretagogue are administered to the mammal daily for a period of at least 24 weeks. In another embodiment, the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, periprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof. In another embodiment, the at least one proton pump inhibitor is given to the mammal in a daily dose of at least 10 mg and the at least one insulin secretagogue is give to the mammal in a daily dose of at least 0.1 mg. In another embodiment, the at least one proton pump inhibitor is given to the mammal in a daily range of at least 5 mg and at most 1,000 mg and the at least one insulin secretagogue is give to the mammal in a daily range of at least 0.1 mg and at most 500 mg. In another embodiment, the composition is in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

In another embodiment, the present invention provides a composition for reducing hemoglobin A1C in a mammal, the composition having a therapeutically effective amount of at least one proton pump inhibitor and a therapeutically effective amount of at least one insulin secretagogue so that at least one proton pump inhibitor and the at least one insulin secretagogue when administered to the mammal causes an increase of the mammal's gastrin concentration levels compared to the mammal's baseline which gastrin concentration levels increase leads to a reduction of the mammal's hemoglobin A1C. In another embodiment, the amount of the at least one proton pump inhibitor and the amount of the at least one insulin secretagogue will reduce hemoglobin A1c in the mammal by at least 1.5%. In another embodiment, the at least one proton pump inhibitor and the at least one insulin secretagogue are administered to the mammal daily for a period of at least 6 weeks and up to 24 weeks. In another embodiment the at least one proton pump inhibitor and the at least one insulin secretagogue are administered to the mammal daily for a period of at least 6 weeks. In another embodiment, the at least one proton pump inhibitor and the at least one insulin secretagogue are administered to the mammal daily for a period of at least 24 weeks. In another embodiment, the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, periprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof. In another embodiment, the at least one proton pump inhibitor is given to the mammal in a daily dose of at least 10 mg and the at least one insulin secretagogue is give to the mammal in a daily dose of at least 0.1 mg. In another embodiment, the at least one proton pump inhibitor is given to the mammal in a daily range of at least 5 mg and at most 1,000 mg and the at least one insulin secretagogue is give to the mammal in a daily range of at least 0.1 mg and at most 500 mg. In another embodiment, the composition is in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

In another embodiment, the present invention provides a method to treat diabetes in a mammal having diabetes, the method comprising administering to the mammal a therapeutically effective amount of at least one proton pump inhibitor and a therapeutically effective amount of at least one insulin secretagogue. In another embodiment, the amount of the at least one proton pump inhibitor and the amount of the at least one insulin secretagogue will reduce hemoglobin A1c in the mammal by at least 1.5%. In another embodiment, the at least one proton pump inhibitor and the at least one insulin secretagogue are administered to the mammal daily for a period of at least 6 weeks and up to 24 weeks. In another embodiment the at least one proton pump inhibitor and the at least one insulin secretagogue are administered to the mammal daily for a period of at least 6 weeks. In another embodiment, the at least one proton pump inhibitor and the at least one insulin secretagogue are administered to the mammal daily for a period of at least 24 weeks. In another embodiment, the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, periprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof. In another embodiment, the at least one proton pump inhibitor is given to the mammal in a daily dose of at least 10 mg and the at least one insulin secretagogue is give to the mammal in a daily dose of at least 0.1 mg. In another embodiment, the at least one proton pump inhibitor is given to the mammal in a daily range of at least 5 mg and at most 1,000 mg and the at least one insulin secretagogue is give to the mammal in a daily range of at least 0.1 mg and at most 500 mg. In another embodiment, the composition is in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

In another embodiment, the present invention provides a method of reducing hemoglobin A1C in a mammal, the method comprising administering to the mammal a therapeutically effective amount of at least one proton pump inhibitor and a therapeutically effective amount of at least one insulin secretagogue. In another embodiment, the amount of the at least one proton pump inhibitor and the amount of the at least one insulin secretagogue will reduce hemoglobin A1c in the mammal by at least 1.5%. In another embodiment, the at least one proton pump inhibitor and the at least one insulin secretagogue are administered to the mammal daily for a period of at least 6 weeks and up to 24 weeks. In another embodiment the at least one proton pump inhibitor and the at least one insulin secretagogue are administered to the mammal daily for a period of at least 6 weeks. In another embodiment, the at least one proton pump inhibitor and the at least one insulin secretagogue are administered to the mammal daily for a period of at least 24 weeks. In another embodiment, the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, periprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof. In another embodiment, the at least one proton pump inhibitor is given to the mammal in a daily dose of at least 10 mg and the at least one insulin secretagogue is give to the mammal in a daily dose of at least 0.1 mg. In another embodiment, the at least one proton pump inhibitor is given to the mammal in a daily range of at least 5 mg and at most 1,000 mg and the at least one insulin secretagogue is give to the mammal in a daily range of at least 0.1 mg and at most 500 mg. In another embodiment, the composition is in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

In another embodiment, the present invention provides a method of optimizing therapeutic efficacy for treatment of diabetes, the method comprising administering at least one proton pump inhibitor and at least one insulin secretagogue to a mammal having diabetes; and determining the level of hemoglobin A1C in the mammal having diabetes, wherein the level of hemoglobin A1C more than 6.2% indicates to increase the amount of the at least one proton pump inhibitor and at least one insulin secretagogue subsequently administered to the mammal and wherein the level of hemoglobin A1C less than 5.8% indicates a need to maintain or reduce the amount of the at least one proton pump inhibitor and at least one insulin secretagogue subsequently administered to the mammal. In another embodiment, the amount of the at least one proton pump inhibitor and the amount of the at least one insulin secretagogue will reduce hemoglobin A1c in the mammal by at least 1.5%. In another embodiment, the at least one proton pump inhibitor and the at least one insulin secretagogue are administered to the mammal daily for a period of at least 6 weeks and up to 24 weeks. In another embodiment the at least one proton pump inhibitor and the at least one insulin secretagogue are administered to the mammal daily for a period of at least 6 weeks. In another embodiment, the at least one proton pump inhibitor and the at least one insulin secretagogue are administered to the mammal daily for a period of at least 24 weeks. In another embodiment, the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, periprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof. In another embodiment, the at least one proton pump inhibitor is given to the mammal in a daily dose of at least 10 mg and the at least one insulin secretagogue is give to the mammal in a daily dose of at least 0.1 mg. In another embodiment, the at least one proton pump inhibitor is given to the mammal in a daily range of at least 5 mg and at most 1,000 mg and the at least one insulin secretagogue is give to the mammal in a daily range of at least 0.1 mg and at most 500 mg. In another embodiment, the composition is in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

### BRIEF DESCRIPTION OF THE DRAWINGS

So that the manner in which the above-recited features, aspects and advantages of the invention, as well as others that will become apparent, are attained and can be understood in detail, more particular description of the invention briefly summarized above can be had by reference to the embodiments thereof that are illustrated in the drawings that form a part of this specification. It is to be noted, however, that the appended drawings illustrate some embodiments of the invention and are, therefore, not to be considered limiting of the invention's scope, for the invention can admit to other equally effective embodiments.

FIG. 1 shows a scatter plot diagram of hemoglobin A1c percent for each patient measured during the course of the trial discussed in Example 4. For all patients, taking PPI resulted in a decrease in percent hemoglobin A1c that was statistically significant.

FIG. 2 shows a scatter plot diagram of hemoglobin A1c percent measured for each patient taking PPI and a insulin secretagogue during the course of the trial discussed in Example 4.

FIG. 3 is a graph that shows glucose levels measured in patients discussed in Example 4. Fasting, one-hour, and two-hour measurements were taken before the patients began taking PPI and then 8 weeks, 16 weeks, and 24 weeks after taking PPI.

FIG. 4 is a graph that shows insulin levels measured in patients discussed in Example 4. Fasting, one-hour, and two-hour measurements were taken before the patients began taking PPI and then 8 weeks, 16 weeks, and 24 weeks after taking PPI.

FIG. 5 is a graph that shows c-peptide levels measured in patients discussed in Example 4. Fasting, one-hour, and two-hour measurements were taken before the patients began taking PPI and then 8 weeks, 16 weeks, and 24 weeks after taking PPI.

FIG. 6 is a graph that shows hemoglobin A1c levels (%) measured in patients discussed in Example 4. Fasting, one-hour, and two-hour measurements were taken before the patients began taking PPI and then 8 weeks, 16 weeks, and 24 weeks after taking PPI. The percent hemoglobin A1c reduction was at least 0.6% from baseline.

FIG. 7 is a graph that shows plasma gastrin levels measured in patients discussed in Example 4. Fasting, one-hour, and two-hour measurements were taken before the patients began taking PPI and then 8 weeks, 16 weeks, and 24 weeks after taking PPI.

FIG. 8 is a graph that shows the effect of co-administration of a secretagogue with a PPI on hemoglobin A1c levels (%) in patients discussed in Example 4. The percent hemoglobin A1c reduction was at least 1.5%.

FIG. 9 is an illustration of a kit containing 30 pills in a blister pack and instructions. Each pill contains at least one proton pump inhibitor and at least one insulin secretagogue.

FIG. 10 is an illustration of a kit containing 60 pills in a blister pack and instructions. Each well has two pills, one pill containing at least one proton pump inhibitor and one pill containing at least one insulin secretagogue.

FIG. 11 is an illustration of a kit containing a transdermal patch and instructions. The patch contains at least one proton pump inhibitor and at least one insulin secretagogue.

### DETAILED DESCRIPTION OF THE PREFERRED EMBODIMENTS

Before describing the embodiments of the present inventions in detail, several terms used in the context of embodiments of the present inventions will be defined. In addition to these terms, others are defined elsewhere in the specification, as necessary. Unless otherwise expressly defined herein, terms of art used in this specification will have their art-recognized meanings.

To more readily facilitate an understanding of the invention, the meanings of terms used herein will become apparent from the context of this specification in view of common usage of various terms and the explicit definitions provided below.

As used herein, the terms “comprising,” “including,” and “such as” are used in their open, non-limiting sense.

The term “about” is used synonymously with the term “approximately.” Illustratively, the use of the term “about” indicates that values slightly outside the cited values, i.e., plus or minus about 0.1 percent to about 10 percent, are also contemplated to be effective and safe. Such dosages are thus encompassed by the scope of the claims reciting the terms “about” and “approximately.”

The phrase “antioxidants” can include, e.g., butylated hydroxytoluene (BHT), sodium ascorbate, and tocopherol.

The term “binders” refers to materials that impart cohesive qualities and can include, e.g., alginic acid and salts thereof; cellulose derivatives such as carboxymethylcellulose, methylcellulose (e.g., Methocel™), hydroxypropylmethylcellulose, hydroxyethylcellulose, hydroxypropylcellulose (e.g., Klucel™), ethylcellulose (e.g., Ethocel™, and microcrystalline cellulose (e.g., Avicel™; microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac™), glucose, dextrose, molasses, mannitol, sorbitol, xylitol (e.g., Xylitab™), and lactose; a natural or synthetic gum such as acacia, tragacanth, ghatti gum, mucilage of isapol husks, polyvinylpyrrolidone (e.g., Polyvidone™, Kollidon™, waxes, and the like.

The term “carrier materials” refer to materials compatible with the proton pump inhibitor and the release profile properties of the desired dosage form. Carrier materials can include, binders, suspending agents, disintegration agents, filling agents, surfactants, solubilizers, stabilizers, lubricants, wetting agents, diluents, and the like.

The term “pharmaceutically compatible carrier materials” can comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

The terms “diffusion facilitators” and “dispersing agents” refer to materials that control the diffusion of an aqueous fluid through a coating. Diffusion facilitators/dispersing agents can include, e.g., hydrophilic polymers, electrolytes, Tween™ 60 or 80, and the like. Combinations of one or more erosion facilitators with one or more diffusion facilitators can also be used in the present invention.

The phrase “diluents” refers to materials that increase bulk of the composition to facilitate compression. Such compounds can include e.g., lactose; starch; mannitol; sorbitol; dextrose; microcrystalline cellulose; dibasic calcium phosphate; dicalcium phosphate dihydrate; tricalcium phosphate; calcium phosphate; anhydrous lactose; spray-dried lactose; pregelatinzed starch; compressible sugar, such as Di-Pac™ (Amstar); mannitol; hydroxy-propylmethylcellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; mannitol; sodium chloride; inositol; bentonite; and the like.

The term “disintegrate” refers to both the dissolution and dispersion of the dosage form when contacted with gastrointestinal fluid.

The term “disintegration agents” is a phrase that refers to compounds or components that facilitate the breakup or disintegration of a substance. Examples of disintegration agents can include a starch, e.g., a natural starch such as corn starch or potato starch, a pregelatinized starch or sodium starch glycolate such as Promogel™.

The term “absorption” refers to the process of movement from the site of administration of a dosage amount toward the systemic circulation, e.g., into the bloodstream of a subject.

An “enteric coating” is a substance that remains substantially intact in the stomach but dissolves and releases the drug once the small intestine is reached. Enteric coatings are generally thought of as “timed release” coatings. Generally, the enteric coating comprises a polymeric material that prevents release in the low pH environment of the stomach but ionizes at a slightly higher pH, typically a pH of 4 or 5, and thus dissolves sufficiently in the small intestines to gradually release the active agent therein.

“Filling agents” can include compounds such as lactose, calcium carbonate, calcium phosphate, dibasic calcium phosphate, calcium sulfate, microcrystalline cellulose, cellulose powder, dextrose; dextrates; dextran, starches, pregelatinized starch, sucrose, xylitol, lactitol, mannitol, sorbitol, sodium chloride, polyethylene glycol, and the like.

“Flavoring agents” or “sweeteners” useful in the pharmaceutical compositions of the present invention can include, e.g., acacia syrup, acesulfame, alitame, anise, apple, 15 aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, 20 lime, lemon cream, monoammonium glyrrhizinate, maltol, mannitol, maple, marshmallow, menthol, mint cream, mixed berry, neohesperidine, neotame, orange, pear, peach, peppermint, peppermint cream, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

The phrase “gastrointestinal fluid” refers to the fluid of stomach secretions of a subject or the saliva of a subject after oral administration of a dosage amount.

“Gastrin” is a naturally occurring 17 amino acid peptide sequence. See www.chemblink.com/structures/10047-33-3.gif as of Nov. 17, 2008, which is hereby incorporated by reference.

“HgbA1c” refers to hemoglobin A1c.

A “measurable serum concentration” or “measurable plasma concentration” refers to the blood serum or blood plasma concentration, typically measured in picograms (pg.), nanograms (ng.) micrograms (meg.) and milligrams (mg.), of a therapeutic agent per ml, dl, or 1 of blood serum.

“Plasma concentration” refers to the concentration of a substance in blood plasma or blood serum of a subject. It is understood that the plasma concentration of a therapeutic agent can vary many-fold between subjects, due to variability with respect to metabolism of therapeutic agents. In accordance with one aspect of the present invention, the plasma concentration of a proton pump inhibitors can vary from subject to subject.

The term “prodrug” refers to a drug or compound in which the pharmacological action results from conversion by metabolic processes within the body. Prodrugs are generally drug precursors that, following administration to a subject and subsequent absorption, are converted to an active, or a more active species via some process, such as conversion by a metabolic pathway. Some prodrugs have a chemical group present on the prodrug which renders it less active and/or confers solubility or some other property to the drug. Once the chemical group has been cleaved and/or modified from the prodrug the active drug is generated. Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak, et al., Am. 1. Physio.l, 269:G210-218 (1995); McLoed, et al., GastroenteroI., 106:405-413 (1994); Hochhaus, et al., Biomed. Chrom., 6:283-286 (1992); J. Larsen and H. Bundgaard, Int. 1. Pharmaceutics, 37, 87 (1987); 1. Larsen et al., Int. J. Pharmaceutics, 47, 103 (1988); Sinkula et al., 1. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987.

The term “solubilizers” can include compounds such as citric acid, succinic acid, fumaric acid, malic acid, tartaric acid, maleic acid, glutaric acid, sodium bicarbonate, sodium carbonate and the like.

The term “stabilizers” can include compounds such as ultra violet stabilizers, antioxidation agents, buffers, acids, and the like.

The term “suspending agents” or “thickening agents” can include compounds such as polyvinylpyrrolidone, polyethylene glycol with a molecular weight of about 300 to about 6000, or about 3350 to about 4000, or about 7000 to about 5400; sodium carboxymethylcellulose; methylcellulose; hydroxy-propylmethylcellulose; polysorbate-80; hydroxyethylcellulose; sodium alginate; gums, such as, e.g., gum tragacanth and gum acacia; guar gum; xanthans, including xanthan gum; sugars; cellulosics, such as, e.g., sodium carboxymethylcellulose, methylcellulose, sodium carboxymethylcellulose, hydroxy-propylmethylcellulose, hydroxyethylcellulose; polysorbate-80; sodium alginate; polyethoxylated sorbitan monolaurate; polyethoxylated sorbitan monolaurate; povidone and the like.

The term “surfactants” can refer to compounds such as sodium lauryl sulfate, sorbitan monooleate, polyoxyethylene sorbitan monooleate, polysorbates, polaxomers, bile salts, glyceryl monostearate, copolymers of ethylene oxide and propylene oxide; and the like.

A “therapeutically effective amount” or “effective amount” is that amount of a pharmaceutical agent to achieve a pharmacological effect. The term “therapeutically effective amount” can include, for example, a prophylactically effective amount. An effective amount” of a proton pump inhibitor is an amount effective to achieve a desired pharmacologic effect or therapeutic improvement without undue adverse side effects.

The words “treat” or “treatment” as used in the context of diabetes or endocrine related disorders refers to the oral treatment of an insulin related disorder or disease associated with a pancreatic disorder wherein some insulin producing cells still remain in the patient. It also refers to patch treatment of the insulin related disorder or disorder of the endocrine system.

It is contemplated that the term “treat” or “treatment” extends to compositions for preventing the endocrine disorder or pancreatic disease from occurring in a subject which may be predisposed to the disorder or disease, but has not yet been diagnosed as having the disorder or disease.

It is contemplated that the term “treat” or “treatment” of diabetes refers to steps taken to inhibit the disorder or disease, e.g., arresting the development of the disorder or disease, relieving the disorder or disease, regression of the disorder or disease, relieving a condition caused by the disease or disorder, or stopping the symptoms of the disease or disorder. Thus, as used herein, the term “treat” is used synonymously with the term “prevent.”

The composition can, in an embodiment, use a carrier for forming an orally ingestible suspension of the at least one proton pump inhibitor. The carrier can be water with a powder formed from at least 4 components which can include: sodium bicarbonate, xylitol, sucrose, sucralose, xanthan gum, ferric oxide, mannitol, docusate sodium, colloidal silicon dioxide, citric acid, sodium citrate, magnesium stearate, artificial flavor, and crospovidone.

The present invention now will be described more fully hereinafter with reference to the accompanying drawings in which embodiments of the invention are shown. This invention may, however, be embodied in many different forms and should not be construed as limited to the exemplary embodiments set forth herein; rather, these embodiments are provided so that this disclosure will be thorough and complete, and will fully convey the scope of the invention to those skilled in the art.

The present embodiments relate to an oral composition for treating type II diabetes and related disorders of the endocrine pancreas of a patient. The embodiments also relate to a patch for continuous delivery of a composition containing at least one proton pump inhibitor for treating type II diabetes.

In an embodiment, the invention is for composition, which can be taking orally or by patch treatment, for treating type II diabetes of a patient, that uses a therapeutically effective amount of at least one proton pump inhibitor for inducing increased serum gastrin concentration levels between about 100 percent to about 3000 percent and creating sustained gastrin concentration level increases over the patient's baseline serum gastrin concentration levels for at least 18 hours after ingestion to reduce the patient's hemoglobin A1c between about 0.5 percent to about 2 percent over a 3 month period by stimulating pancreatic beta cells and increasing the mass of pancreatic beta cells in a patient to produce increased insulin.

The composition is particularly useful when patient baseline serum gastrin concentration levels are initially is between about 15 to about 150 picograms/ml and after orally ingesting the dosage increase to between about 100 to about 3000 picograms/ml.

The proton pump inhibitor increases serum gastrin concentration levels in the patient between about 100 percent to about 3000 percent, for a time period substantially equivalent to the time period during treatment of the patient with the therapeutically effective amounts of the proton pump inhibitor. If orally administered for a period of months, at least every 24 hours, the composition increases the patient's serum gastrin concentration above a baseline so that the hemoglobin A1c percentage decreases between about 0.2 percent to about 3 percent.

A benefit of the invention is that it increases serum gastrin concentrations without gastrointestinal consequences including nausea, vomiting, diarrhea and electrolyte disturbances which could lead to heart attack or stroke. The composition prevents dehydration of a patient and prevents placing a diabetic patient in a weakened condition. Another benefit of the invention is that the dosage amounts can be varied to accommodate small children and adults. Another benefit of the invention is that no additional anti-inflammatory medicines need to be included with the treatment.

An additional benefit of the invention is that no incretin mimetics are needed, in this therapy, the composition is not injectable, which for diabetics reduces risk of infection at the injection site, and eliminates tissue injury, the common creation of “black and blue” spots on diabetics which are slow to heal. For the patch version of the invention no incretin mimetics are needed, however, it is contemplated that components that increase the transdermal absorption can be used in combination with the proton pump inhibitor for effective delivery. In an embodiment, it is contemplated that this composition can be topically delivered using creams or gels.

Proton pump inhibitors (PPI) can be a class of acid-labile pharmaceutical compounds that block gastric acid secretion pathways. Proton pump inhibitors can include, omeprazole (Prilosec™), lansoprazole (Prevacid™), esomeprazole (Nexium™), rabeprazole (Aciphex™), pantoprazole (Protonix™), pariprazole, tenatoprazole, and leminoprazole. The drugs of this class suppress gastrointestinal acid secretion by the specific inhibition of the H+/K+-ATPase enzyme system (proton pump) at the secretory surface of the gastrointestinal parietal cell.

Most proton pump inhibitors are susceptible to acid degradation and, as such, are rapidly destroyed in an acidic pH environment in the stomach. Therefore, proton pump inhibitors are often administered as enteric-coated dosage forms in order to permit release of the drug in the duodenum after having passed through the stomach. Omeprazole, a substituted bicyclic aryl-imidazole, 5-methoxy-2-[(4-methoxy-3,5-dimethyl-2-pyridinyl)methyl]sulfinyl-1H-benzimidazole, is a proton pump inhibitor that inhibits gastrointestinal acid secretion.

Non-enteric coated pharmaceutical compositions which facilitate immediate release of the pharmaceutically active ingredient into the stomach and permit stomach uptake of pharmaceutical agents can be used herein.

Proton pump inhibitors are typically prescribed for short-term treatment of active duodenal ulcers, gastrointestinal ulcers, gastro esophageal reflux disease (GERD), severe erosive esophagitis, poorly responsive symptomatic GERD, and pathological hypersecretory conditions such as Zollinger Ellison syndrome. It is unexpected to use these inhibitors orally to increase pancreatic beta cell mass in a patient or to stimulate pancreatic cell insulin production. Similarly, use of proton pump inhibitors for a patch is unexpected.

It is believed that omeprazole, lansoprazole and other proton pump inhibiting agents reduce gastrointestinal acid production by inhibiting H+/K+-ATPase of the parietal cell, which is the final common pathway for gastrointestinal acid secretion. See, e.g., Fellenius et al., Substituted Benzimidazoles Inhibit Gastrointestinal Acid Secretion by Blocking H+/K+-ATPase, Nature, 290: 159-161 (1981); Wallmark et al., The Relationship Between Gastrointestinal Acid Secretion and Gastrointestinal H+/K+-ATPase Activity, J. Biol. Chem., 260: 13681-13684 (1985); and Fryklund et al., Function and Structure of Parietal Cells After H+/K+-ATPase Blockade, Am. 1. Physiol., 254 (1988). Use for diabetes without additional additives has not been suggested in these references.

Proton pump inhibitors have the ability to act as weak bases which reach parietal cells from the blood and diffuse into the secretory canaliculi. The drugs become protonated. The protonated compound can then rearrange to form a sulfonamide which can covalently interact with sulfhydryl groups at critical sites in the extra cellular (luminal) domain of the membrane-spanning H+/K+-ATPase. See, e.g., Hardman et al., Goodman & Gilman's The Pharmacological Basis of Therapeutics, 907 (9th ed. 1996). As such, proton pump inhibitors are prodrugs that must be activated within parietal cells to be effective. The specificity of the effects of proton pump inhibiting agents is also dependent upon: (a) the selective distribution of H+/K+-ATPase; (b) the requirement for acidic conditions to catalyze generation of the reactive inhibitor; and (c) the trapping of the protonated drug and the cationic sulfonamide within the acidic canaliculi adjacent to the target enzyme.

Proton pump inhibitors can include, but are not limited to hydroxyomeprazole, esomeprazole, tenatoprazole, lansoprazole, pantoprazole, rabeprazole, dontoprazole, habeprazole, periprazole, ransoprazole, pariprazole, leminoprazole; or a free base, free acid, salt, hydrate, ester, amide, enantiomer, isomer, tautomer, polymorph, or prodrug thereof.

The composition can have between about 10 mg to about 3000 mg of proton pump inhibitor, the composition can further have about 5 mg, 10 mg, about 15 mg, about 20 mg, 20 about 30 mg, about 40 mg, about 50 mg, about 60 mg, about 80 mg, about 160 mg or about 320 mg of the proton pump inhibitor.

The composition which is for oral administration to a patient provides a pharmacokinetic profile such that at least about 50 percent of total area under serum concentration time curve (AVe) for the proton pump inhibitor occurs within about 2 hours after administration of a single dose of the composition to the patient. This composition can be used for humans or for horses, dogs, or other mammals over about 15 pounds.

An embodiment contemplates at least some of the proton pump inhibitor can be encapsulated, such as with microencapsulated proton pump inhibitors to create an efficient delayed delivery of the composition, in effect, a timed release of the proton pump inhibitor though the gastric system. The coated embodiment of the composition allows more even absorption of the material for more even stimulation to form beta cells.

Encapsulation materials can include oatmeal, cellulose hydroxypropyl ethers, low-substituted hydroxypropyl ethers, cellulose hydroxypropyl methyl ethers, methylcellulose polymers, ethylcelluloses and mixtures thereof, polyvinyl alcohol, hydroxyethylcelluloses, carboxymethylcelluloses, salts of carboxymethylcelluloses, polyvinyl alcohol, polyethylene glycol co-polymers, monoglycerides, triglycerides, polyethylene glycols, modified food starch, acrylic polymers, mixtures of acrylic polymers with cellulose ethers, cellulose acetate phthalate, sepifilms, cyclodextrins; and mixtures thereof.

Examples of cellulose hydroxypropyl ether can include Klucel™ or Nisso™. The cellulose hydroxypropyl methyl ether can be Seppifilm™, Pharmacoat™., Metolose™, Opadry™, PrimaFlo™, BenecelMP824, or BenecelMP843. Methocel™, Benecel™, or Metolose™ can be used. Ethylcellulose or mixture thereof can be used such as, Ethocel™ Benecel™, and Celacal.

The polyvinyl alcohol can be, but is not limited to, Opadry™. The acrylic polymers or mixtures thereof can include, but are not limited to, Eudragits™. Other materials can be used as micro-encapsulates that not only provide timed release benefits but also enhance the shelf-life of the pharmaceutical composition. Coatings that enhance the shelf-life of the pharmaceutical The polyvinyl alcohol can be, but is not limited to, Opadry™. The acrylic polymers or mixtures thereof can include, but are not limited to, Eudragits™. Other materials can be used as micro-encapsulates that not only provide timed release benefits but also enhance the shelf-life of the pharmaceutical composition. Coatings that enhance the shelf-life of the pharmaceutical composition can further include other compatible materials such as an antioxidant, a plasticizer, a buffering agent, and mixtures thereof.

The composition is intended to treat type II diabetes wherein the patient fails to produce adequate insulin in response to defined levels of plasma glucose. The composition is also intended to treat type II diabetes where the patient has a tissue resistance to insulin effect, or an impaired insulin secretion, or a reduced pancreatic beta cell mass as compared to patients without the disorder, normal patents of the same age and condition.

In an embodiment, the composition can include a second therapeutically effective amount of at least one insulin secretagogue with the proton pump inhibitor. In an embodiment, the insulin secretagogue can be a sulfonylurea, neteglinide, and repaglinide. The sulfonylurea can be acetohexamide, chlorpropamide, tolbutamide, tolazamide, glipizide, gliclazide, glibenclamide, gliquidone, glyclopyramide, glimepiride, or a free base, free acid, salt, hydrate, ester, amide, enantiomer, isomer, tautomer, polymorph, or prodrug, and combinations thereof.

It can be contemplated that the composition can include the following dosing combinations of proton pump inhibitors (PPI) and other oral diabetes medications.

In another embodiment of the composition, the composition can include a second therapeutically effective amount of at least one thiazolidinedione with the proton pump inhibitor. The thiazolidinedione can be a pioglitazone, a rosiglitazone or combinations thereof.

Still another embodiment of the composition can include a metformin.

Another embodiment of the composition can contemplate including at least one dipeptidyl peptidase IV inhibitors. One or more dipeptidyl peptidase IV inhibitors can be used in the formulation, such as sitagliptin, vildagliptin, saxagliptin, denagliptin, or combinations thereof.

In still another embodiment of the invention, at least two therapeutically effective amounts of a dipeptidyl peptidase IV inhibitors, metformin, a thiazolidinedione, and a sulfonylurea can be used with the first proton pump inhibitor.

For a dosage of Esomeprazole and Pioglitazone: 20 mg/15 mg, 1 po daily, 20 mg/15 mg, 1 po bid, 20 mg/30 mg, 1 po daily, 20 mg/7.5 mg, 1 po bid, 20 mg/45 mg, 1 po daily, 20 mg/22.5 mg, 1 po bid; 40 mg/15 mg, 1 po daily, 40 mg/15 mg, 1 po bid, 40 mg/30 mg, 1 po daily, 40 mg/7.5 mg, 1 po bid, 40 mg/45 mg, 1 po daily, 40 mg/22.5 mg, 1 po bid; 80 mg/15 mg, 1 po daily, 80 mg/15 mg, 1 po bid, 80 mg/30 mg, 1 po daily, 80 mg/7.5 mg, 1 po bid, 80 mg/45 mg, 1 po daily, 80 mg/22.5 mg, 1 po bid; 160 mg/15 mg, 1 po daily, 160 mg/15 mg, 1 po bid, 160 mg/30 mg, 1 po daily, 160 mg/7.5 mg, 1 po bid, 160 mg/45 mg, 1 po daily, 160 mg/22.5 mg, 1 po bid.

For a dosage of Esomeprazole and Metformin (esomeprazole/metformin immediate release (IR) and extended release (ER): 20 mg/500 mg IR 1 po daily, 20 mg/500 mg IR 5 1 po bid, 40 mg/500 mg IR, 1 po daily, 40 mg/500 mg IR 1 po bid, 20 mg/750 mg IR, 1 po daily, 20 mg/750 mg IR 1 po bid, 40 mg/750 mg IR, 1 po daily, 40 mg/750 mg IR 1 po bid, 20 mg/850 mg IR, 1 po daily, 20 mg/850 mg IR 1 po bid, 40 mg/850 mg IR, 1 po daily, 40 mg/850 mg IR 1 po bid, 20 mg/1000 mg IR, 1 po daily, 20 mg/1000 mg IR 1 po bid, 40 mg/1000 mg IR, 1 po daily, 40 mg/1000 mg IR 1 po bid, 80 mg/500 mg IR, 1 po 10 daily, 80 mg/500 mg IR 1 po bid, 80 mg/750 mg IR, 1 po daily, 80 mg/750 mg IR 1 po bid, 80 mg/850 mg IR, 1 po daily, 80 mg/850 mg IR 1 po bid, 80 mg/1000 mg IR, 1 po daily, 80 mg/1000 mg IR 1 po bid, 160 mg/500 mg IR, 1 po daily, 160 mg/500 mg IR 1 po bid, 160 mg/750 mg IR, 1 po daily, 160 mg/750 mg IR 1 po bid, 160 mg/850 mg IR, 1 po daily, 160 mg/850 mg IR 1 po bid, 160 mg/1000 mg IR, 1 po daily, 160 mg/1000 mg 15 IR 1 po bid. Extended release: 20 mg/500 mg ER, 1 po daily, 20 mg/500 mg ER 2 po daily, 20 mg/500 mg ER 3 po daily, 20 mg/500 mg ER 4 po daily, 20 mg/750 mg ER, 1 po daily, 20 mg/750 mg ER 2 po daily, 20 mg/750 mg ER 3 po daily, 20 mg/850 mg ER, 1 po daily, 20 mg/850 mg ER 2 po daily, 20 mg/1000 mg, 1 po daily, 20 mg/1000 mg 2 po daily; 40 mg/500 mg ER, 1 po daily, 40 mg/500 mg ER 2 po daily, 40 mg/500 mg ER 20 3 po daily, 40 mg/500 mg ER 4 po daily, 40 mg/750 mg ER, 1 po daily, 40 mg/750 mg ER 2 po daily, 40 mg/750 mg ER 3 po daily, 40 mg/850 mg ER, 1 po daily, 40 mg/850 mg ER 2 po daily, 40 mg/1000 mg ER, 1 po daily, 40 mg/1000 mg ER 2 po daily; 80 mg/500 mg ER, 1 po daily, 80 mg/500 mg ER 2 po daily, 80 mg/500 mg ER 3 po daily, 80 mg/500 mg ER 4 po daily, 80 mg/750 mg ER, 1 po daily, 80 mg/750 mg ER2 po daily, 80 mg/750 mg ER 3 po daily, 80 mg/850 mg ER, 1 po daily, 80 mg/850 mg ER 2 po daily, 80 mg/1000 mg ER, 1 po daily, 80 mg/1000 mg ER 2 po daily.

For a dosage of Esomeprazole and Pioglitazone and Metformin combinations

(esomeprazole/pioglitazone/metformin): 20 mg/15 mg/500 mg, 1 po daily, 20 mg/15 mg/500 mg 1 po bid, 20 mg/15 mg/850 mg, 1 po daily, 20 mg/15 mg/850 mg 1 po bid, 20 mg/15 mg/1000 mg, 1 po daily, 20 mg/15 mg/1000 mg 1 po bid, 40 mg/15 mg/500 mg, 1 po daily, 40 mg/15 mg/500 mg 1 po bid, 40 mg/15 mg/850 mg, 1 po daily, 40 mg/15 mg/850 mg 1 po bid, 40 mg/15 mg/1000 mg, 1 po daily, 40 mg/15 mg/1000 mg 1 po bid, 80 mg/15 mg/500 mg, 1 po daily, 80 mg/15 mg/500 mg 1 po bid, 80 mg/15 mg/850 mg, 1 po daily, 80 mg/15 mg/850 mg 1 po bid, 80 mg/15 mg/1000 mg, 1 po daily, 80 mg/15 mg/1000 mg 1 po bid, 160 mg/15 mg/500 mg, 1 po daily, 160 mg/15 mg/500 mg 1 po bid, 160 mg/15 mg/850 mg, 1 po daily, 160 mg/15 mg/850 mg 1 po bid, 160 mg/15 mg/1000 mg, 1 po daily, 160 mg/15 mg/1000 mg 1 po bid.

For a dosage of Esomeprazole and Rosiglitazone (esomeprazole/rosiglitazone): 20 mg/4 mg, 1 po daily, 20 mg/4 mg 1 po bid, 20 mg/2 mg, 1 po daily, 20 mg/2 mg 1 po bid, 20 mg/8 mg, 1 po daily, 40 mg/2 mg, 1 po daily, 40 mg/2 mg 1 po bid, 40 mg/4 mg, 1 po daily, 40 mg/4 mg 1 po bid, 40 mg/8 mg, 1 po daily, 80 mg/4 mg, 1 po daily, 80 mg/4 mg 1 po bid, 80 mg/8 mg, 1 po daily, 160 mg/4 mg, 1 po daily, 160 mg/4 mg 1 po bid, 160 mg/2 mg, 1 po daily, 160 mg/2 mg 1 po bid, 160 mg/8 mg, 1 po daily.

For a dosage of Esomeprazole and Rosiglitazone and Metformin (IR and ER) combinations (esomeprazole/rosiglitazone/metformin): 20 mg/2 mg/500 mg, 1 po daily, 20 mg/2 mg/500 mg 1 po bid, 20 mg/2 mg/850 mg, 1 po daily, 20 mg/2 mg/850 mg 1 po bid, 20 mg/2 mg/1000 mg, 1 po daily, 20 mg/2 mg/1000 mg 1 po bid, 20 mg/4 mg/500 mg, 1 po daily, 20 mg/4 mg/500 mg 1 po bid, 20 mg/4 mg/850 mg, 1 po daily, 20 mg/4 mg/850 mg 1 po bid, 20 mg/4 mg/1000 mg, 1 po daily, 20 mg/4 mg/1000 mg 1 po bid, 40 mg/2 mg/500 mg, 1 po daily, 40 mg/2 mg/500 mg 1 po bid, 40 mg/2 mg/850 mg, 1 po daily, 40 mg/2 mg/850 mg 1 po bid, 40 mg/2 mg/1000 mg, 1 po daily, 40 mg/2 mg/1000 mg 1 po bid, 40 mg/4 mg/500 mg, 1 po daily, 40 mg/4 mg/500 mg 1 po bid, 40 mg/4 mg/850 mg, 1 po daily, 40 mg/4 mg/850 mg 1 po bid, 40 mg/4 mg/1000 mg, 1 po daily, 40 mg/4 mg/1000 mg 1 po bid, 80 mg/2 mg/500 mg, 1 po daily, 80 mg/2 mg/500 mg 1 po bid, 80 mg/2 mg/850 mg, 1 po daily, 80 mg/2 mg/850 mg 1 po bid, 80 mg/2 mg/1000 mg, 1 po daily, 80 mg/2 mg/1000 mg 1 po bid, 80 mg/4 mg/500 mg, 1 po daily, 80 mg/4 mg/500 mg 1 po bid, 80 mg/4 mg/850 mg, 1 po daily, 80 mg/4 mg/850 mg 1 po bid, 80 mg/4 mg/1000 mg, 1 po daily, 80 mg/4 mg/1000 mg 1 po bid, 160 mg/2 mg/500 mg, 1 po daily, 160 mg/2 mg/500 mg 1 po bid, 160 mg/2 mg/850 mg, 1 po daily, 160 mg/2 mg/850 mg 1 po bid, 160 mg/2 mg/1000 mg, 1 po daily, 160 mg/2 mg/1000 mg 1 po bid, 160 mg/4 mg/500 mg, 1 po daily, 160 mg/4 mg/500 mg 1 po bid, 160 mg/4 mg/850 mg, 1 po daily, 160 mg/4 mg/850 mg 1 po bid, 160 mg/4 mg/1000 mg, 1 po daily, 160 mg/4 mg/1000 mg 1 po bid.

For a dosage of Esomeprazole and Sitagliptan (esomeprazole/sitagliptan): 20 mg/50 mg, 1 po daily, 20 mg/50 mg 1 po bid, 40 mg/50 mg, 1 po daily, 40 mg/50 mg 1 po bid, 80 mg/50 mg, 1 po daily, 80 mg/50 mg 1 po bid, 20 mg/100 mg, 1 po daily, 40 mg/100 mg, 1 po daily, 80 mg/100 mg, 1 po daily, 160 mg/50 mg, 1 po daily, 160 mg/50 mg 1 po bid, 160 mg/100 mg, 1 po daily.

For a dosage of Esomeprazole and Sitagliptan and Metformin (IR and ER) combinations (esomeprazole/sitagliptan/metformin): 20 mg/50 mg/500 mg, 1 po daily, 15 20 mg/50 mg/500 mg 1 po bid, 20 mg/50 mg/850 mg, 1 po daily, 20 mg/50 mg/850 mg 1 po bid, 20 mg/50 mg/1000 mg, 1 po daily, 20 mg/50 mg/1000 mg 1 po bid, 40 mg/50 mg/500 mg, 1 po daily, 40 mg/50 mg/500 mg 1 po bid, 40 mg/50 mg/850 mg, 1 po daily, 40 mg/50 mg/850 mg 1 po bid, 40 mg/50 mg/1000 mg, 1 po daily, 40 mg/50 mg/1000 mg 1 po bid, 40 mg/100 mg/500 mg, 1 po daily, 40 mg/100 mg/850 mg, 20 1 po daily, 40 mg/100 mg/1000 mg, 1 po daily, 40 mg/100 mg/2000 mg 1 po daily, 800 mg/50 mg/500 mg, 1 po daily, 80 mg/50 mg/500 mg 1 po bid, 80 mg/50 mg/850 mg, 1 po daily, 80 mg/50 mg/850 mg 1 po bid, 80 mg/50 mg/1000 mg, 1 po daily, 80 mg/50 mg/1000 mg 1 po bid, 80 mg/100 mg/500 mg, 1 po daily, 80 mg/100 mg/850 mg, 1 po daily, 80 mg/100 mg/1000 mg, po daily, 80 mg/100 mg/2000 mg 1 po daily, 160 mg/50 mg/500 mg, 1 po daily, 160 mg/50 mg/500 mg 1 po bid, 160 mg/50 mg/850 mg, 1 po daily, 160 mg/50 mg/850 mg 1 po bid, 160 mg/50 mg/1000 mg, 1 po daily, 160 mg/50 mg/1000 mg 1 po bid, 160 mg/100 mg/500 mg, 1 po daily, 160 mg/100 mg/850 mg, 1 po daily, 160 mg/100 mg/1000 mg, 1 po daily, 160 mg/100 mg/2000 mg 1 po daily.

For a dosage of Esomeprazole and glimiperide (esomeprazole/glimiperide): 20 mg/1 mg, 1 po daily, 20 mg/1 mg 1 po bid, 20 mg/2 mg, 1 po daily, 20 mg/2 mg, 1 po bid, 20 mg/3 mg, 1 po daily, 20 mg/3 mg, 1 po bid, 20 mg/4 mg, 1 po daily, 20 mg/4 mg, 1 po bid, 20 mg/6 mg, 1 po daily, 20 mg/8 mg, 1 po daily, 40 mg/1 mg, 1 po daily, 40 mg/1 mg, 1 po bid, 40 mg/2 mg, 1 po daily, 40 mg/2 mg, 1 po bid, 40 mg/3 mg, 1 po daily, 40 mg/3 mg, 1 po bid, 40 mg/4 mg, 1 po daily, 40 mg/4 mg, 1 po bid, 40 mg/6 mg, 1 po daily, 40 mg/8 mg, 1 po daily, 80 mg/1 mg, 1 po daily, 80 mg/1 mg, 1 po bid, 80 mg/2 mg, 1 po daily, 80 mg/2 mg, 1 po bid, 80 mg/3 mg, 1 po daily, 80 mg/3 mg, 1 po bid, 80 mg/4 mg, 1 po daily, 80 mg/4 mg, 1 po bid, 80 mg/6 mg, 1 po daily, 80 mg/8 mg, 1 po daily, 160 mg/1 mg, 1 po daily, 160 mg/1 mg, 1 po bid, 160 mg/2 mg, 1 po daily, 160 mg/2 mg, 1 po bid, 160 mg/3 mg, 1 po daily, 160 mg/3 mg, 1 po bid, 160 mg/4 mg, 1 po daily, 160 mg/4 mg, 1 po bid, 160 mg/6 mg, 1 po daily, 160 mg/8 mg, 1 po daily.

For a dosage of Esomeprazole and glimiperide and pioglitazone combinations (esomeprazole/glimiperide/pioglitazone): 20 mg/1 mg/15 mg, 1 po daily, 20 mg/1 mg/15 mg 1 po bid, 20 mg/2 mg/15 mg, 1 po daily, 20 mg/2 mg/15 mg, 1 po bid, 20 mg/3 mg/15 mg, 1 po daily, 20 mg/3 mg/15 mg, 1 po bid, 20 mg/4 mg/15 mg, 1 po daily, 20 mg/4 mg/15 mg, 1 po bid, 20 mg/6 mg/15 mg, 1 po daily, 20 mg/6 mg/30 mg, 1 po daily, 20 mg/6 mg/45 mg, 1 po daily, 20 mg/8 mg/15 mg, 1 po daily, 20 mg/8 mg/30 mg, 1 po daily, 20 mg/8 mg/45 mg, 1 po daily, 40 mg/1 mg/15 mg, 1 po daily, 40 mg/1 mg/15 mg, 1 po bid, 40 mg/2 mg/15 mg, 1 po daily, 40 mg/2 mg/15 mg, 1 po bid, 40 mg/3 mg/15 mg, 1 po daily, 40 mg/3 mg/15 mg, 1 po bid, 40 mg/4 mg/15 mg, 1 po daily, 40 mg/4 mg/15 mg, 1 po bid, 40 mg/6 mg/15 mg, 1 po daily, 40 mg/6 mg/30 mg, 1 po daily, 40 mg/6 mg/45 mg, 1 po daily, 40 mg/8 mg/15 mg, 1 po daily, 40 mg/8 mg/30 mg, 1 po daily, 40 mg/8 mg/45 mg, 1 po daily, 80 mg/1 mg/15 mg, 1 po daily, 80 mg/1 mg/15 mg, 1 po bid, 80 mg/2 mg/15 mg, 1 po daily, 80 mg/2 mg/15 mg, 1 po bid, 80 mg/3 mg/15 mg, 1 po daily, 80 mg/3 mg/15 mg, 1 po bid, 80 mg/4 mg/15 mg, 1 po daily, 80 mg/4 mg/15 mg, 1 po bid, 80 mg/6 mg/15 mg, 1 po daily, 80 mg/6 mg/30 mg, 1 po daily, 80 mg/6 mg/30 mg, 1 po daily, 80 mg/8 mg/15 mg, 1 po daily, 80 mg/8 mg/30 mg, 1 po daily, 80 mg/8 mg/45 mg, 1 po daily, 160 mg/1 mg/15 mg, 1 po daily, 160 mg/1 mg/15 mg, 1 po bid, 160 mg/2 mg/15 mg, 1 po daily, 160 mg/2 mg/15 mg, 1 po bid, 160 mg/3 mg/15 mg, 1 po daily, 160 mg/3 mg/15 mg, 1 po bid, 160 mg/4 mg/15 mg, 1 po daily, 160 mg/4 mg/15 mg, 1 po bid, 160 mg/6 mg/15 mg, 1 po daily, 160 mg/6 mg/30 mg, 1 po daily, 160 mg/6 mg/45 mg, 1 po daily, 160 mg/8 mg/15 mg, 1 po daily, 160 mg/8 mg/30 mg, 1 po daily, 160 mg/8 mg/45 mg, 1 po daily.

For a dosage of Esomeprazole and glimiperide and metformin combinations (esomeprazole/glimiperide/metformin): 20 mg/1 mg/500 mg, 1 po daily, 20 mg/1 mg/500 mg 1 po bid, 20 mg/2 mg/500 mg, 1 po daily, 20 mg/2 mg/500 mg, 1 po bid, 20 mg/3 mg/500 mg, 1 po daily, 20 mg/3 mg/500 mg, 1 po bid, 20 mg/4 mg/500 mg, 1 po daily, 20 mg/4 mg/500 mg, 1 po bid, 20 mg/6 mg/500 mg, 1 po daily, 20 mg/8 mg/500 mg, 1 po daily, 20 mg/1 mg/750 mg, 1 po daily, 20 mg/1 mg/750 mg 1 po bid, 20 mg/2 mg/750 mg, 1 po daily, 20 mg/2 mg/750 mg, 1 po bid, 20 mg/3 mg/750 mg, 1 po daily, 20 mg/3 mg/750 mg, 1 po bid, 20 mg/4 mg/750 mg, 1 po daily, 20 mg/4 mg/750 mg, 1 po bid, 20 mg/6 mg/750 mg, 1 po daily, 20 mg/8 mg/750 mg, 1 po daily, 20 mg/1 mg/850 mg, 1 po daily, 20 mg/1 mg/850 mg 1 po bid, 20 mg/2 mg/850 mg, 1 po daily, 20 mg/2 mg/850 mg, 1 po bid, 20 mg/3 mg/850 mg, 1 po daily, 20 mg/3 mg/850 mg, 1 po bid, 20 mg/4 mg/850 mg, 1 po daily, 20 mg/4 mg/850 mg, 1 po bid, 20 mg/6 mg/850 mg, 1 po daily, 20 mg/8 mg/850 mg, 1 po daily, 20 mg/1 mg/1000 mg, 1 po daily, 20 mg/1 mg/1000 mg 1 po bid, 20 mg/2 mg/1000 mg, 1 po daily, 20 mg/2 mg/1000 mg, 1 po bid, 20 mg/3 mg/1000 mg, 1 po daily, 20 mg/3 mg/1000 mg, 1 po bid, 20 mg/4 mg/1000 mg, 1 po daily, 20 mg/4 mg/1000 mg, 1 po bid, 20 mg/6 mg/1000 mg, 1 po daily, 20 mg/8 mg/1000 mg, 1 po daily, 40 mg/1 mg/500 mg, 1 po daily, 40 mg/1 mg/500 mg 1 po bid, 40 mg/2 mg/500 mg, 1 po daily, 40 mg/2 mg/500 mg, 1 po bid, 40 mg/3 mg/500 mg, 1 po daily, 40 mg/3 mg/500 mg, 1 po bid, 40 mg/4 mg/500 mg, 1 po daily, 40 mg/4 mg/500 mg, 1 po bid, 40 mg/6 mg/500 mg, 1 po daily, 40 mg/8 mg/500 mg, 1 po daily, 40 mg/1 mg/750 mg, 1 po daily, 40 mg/1 mg/750 mg 1 po bid, 40 mg/2 mg/750 mg, 1 po daily, 40 mg/2 mg/750 mg, 1 po bid, 40 mg/3 mg/750 mg, 1 po daily, 40 mg/3 mg/750 mg, 1 po bid, 40 mg/4 mg/750 mg, 1 po daily, 40 mg/4 mg/750 mg, 1 po bid, 40 mg/6 mg/750 mg, 1 po daily, 40 mg/8 mg/750 mg, 1 po daily, 40 mg/1 mg/850 mg, 1 po daily, 40 mg/1 mg/850 mg 1 po bid, 40 mg/2 mg/850 mg, 1 po daily, 40 mg/2 mg/850 mg, 1 po bid, 40 mg/3 mg/850 mg, 1 po daily, 40 mg/3 mg/850 mg, 1 po bid, 40 mg/4 mg/850 mg, 1 po daily, 40 mg/4 mg/850 mg, 1 po bid, 40 mg/6 mg/850 mg, 1 po daily, 40 mg/8 mg/850 mg, 1 po daily, 40 mg/1 mg/1000 mg, 1 po daily, 40 mg/1 mg/1000 mg 1 po bid, 40 mg/2 mg/1000 mg, 1 po daily, 40 mg/2 mg/1000 mg, 1 po bid, 40 mg/3 mg/1000 mg, 1 po daily, 40 mg/3 mg/1000 mg, 1 po bid, 40 mg/4 mg/1000 mg, 1 po daily, 40 mg/4 mg/1000 mg, 1 po bid, 40 mg/6 mg/1000 mg, 1 po daily, 40 mg/8 mg/1000 mg, 1 po daily, 80 mg/1 mg/500 mg, 1 po daily, 80 mg/1 mg/500 mg 1 po bid, 80 mg/2 mg/500 mg, 1 po daily, 80 mg/2 mg/500 mg, 1 po bid, 80 mg/3 mg/500 mg, 1 po daily, 80 mg/3 mg/500 mg, 1 po bid, 80 mg/4 mg/500 mg, 1 po daily, 80 mg/4 mg/500 mg, 1 po bid, 80 mg/6 mg/500 mg, 1 po daily, 80 mg/8 mg/500 mg, 1 po daily, 80 mg/1 mg/750 mg, 1 po daily, 80 mg/1 mg/750 mg 1 po bid, 80 mg/2 mg/750 mg, 1 po daily, 80 mg/2 mg/750 mg, 1 po bid, 80 mg/3 mg/750 mg, 1 po daily, 80 mg/3 mg/750 mg, 1 po bid, 80 mg/4 mg/750 mg, 1 po daily, 80 mg/4 mg/750 mg, 1 po bid, 80 mg/6 mg/750 mg, 1 po daily, 80 mg/8 mg/750 mg, 1 po daily, 80 mg/1 mg/850 mg, 1 po daily, 80 mg/1 mg/850 mg 1 po bid, 80 mg/2 mg/850 mg, 1 po daily, 80 mg/2 mg/850 mg, 1 po bid, 80 mg/3 mg/850 mg, 1 po daily, 80 mg/3 mg/850 mg, 1 po bid, 80 mg/4 mg/850 mg, 1 po daily, 80 mg/4 mg/850 mg, 1 po bid, 80 mg/6 mg/850 mg, 1 po daily, 80 mg/8 mg/850 mg, 1 po daily, 80 mg/1 mg/1000 mg, 1 po daily, 80 mg/1 mg/1000 mg 1 po bid, 80 mg/2 mg/1000 mg, 1 po daily, 80 mg/2 mg/1000 mg, 1 po bid, 80 mg/3 mg/1000 mg, 1 po daily, 80 mg/3 mg/1000 mg, 1 po bid, 80 mg/4 mg/1000 mg, 1 po daily, 80 mg/4 mg/1000 mg, 1 po bid, 80 mg/6 mg/1000 mg, 1 po daily, 80 mg/8 mg/1000 mg, 1 po daily, 160 mg/1 mg/500 mg, 1 po daily, 160 mg/1 mg/500 mg 1 po bid, 160 mg/2 mg/500 mg, 1 po daily, 160 mg/2 mg/500 mg, 1 po bid, 160 mg/3 mg/500 mg, 1 po daily, 160 mg/3 mg/500 mg, 1 po bid, 160 mg/4 mg/500 mg, 1 po daily, 160 mg/4 mg/500 mg, 1 po bid, 160 mg/6 mg/500 mg, 1 po daily, 160 mg/8 mg/500 mg, 1 po daily, 160 mg/1 mg/750 mg, 1 po daily, 160 mg/1 mg/750 mg 1 po bid, 160 mg/2 mg/750 mg, 1 po daily, 160 mg/2 mg/750 mg, 1 po bid, 160 mg/3 mg/750 mg, 1 po daily, 160 mg/3 mg/750 mg, 1 po bid, 160 mg/4 mg/750 mg, 1 po daily, 160 mg/4 mg/750 mg, 1 po bid, 160 mg/6 mg/750 mg, 1 po daily, 160 mg/8 mg/750 mg, 1 po daily, 160 mg/1 mg/850 mg, 1 po daily, 160 mg/1 mg/850 mg 1 po bid, 160 mg/2 mg/850 mg, 1 po daily, 160 mg/2 mg/850 mg, 1 po bid, 160 mg/3 mg/850 mg, 1 po daily, 160 mg/3 mg/850 mg, 1 po bid, 160 mg/4 mg/850 mg, 1 po daily, 160 mg/4 mg/850 mg, 1 po bid, 5 160 mg/6 mg/850 mg, 1 po daily, 160 mg/8 mg/850 mg, 1 po daily, 160 mg/1 mg/1000 mg, 1 po daily, 160 mg/1 mg/1000 mg 1 po bid, 160 mg/2 mg/1000 mg, 1 po daily, 160 mg/2 mg/1000 mg, 1 po bid, 160 mg/3 mg/1000 mg, 1 po daily, 160 mg/3 mg/1000 mg, 1 po bid, 160 mg/4 mg/1000 mg, 1 po daily, 160 mg/4 mg/1000 mg, 1 po bid, 160 mg/6 mg/1000 mg, 1 po daily, 160 mg/8 mg/1000 mg, 1 po daily.

The composition can further contemplate having at least one buffering agent in an amount sufficient to increase gastric fluid pH or a pH that prevents acid degradation of at least some of the proton pump inhibitor.

The buffering agent can be an alkaline earth metal salt or a Group IA metal selected from a bicarbonate salt of a Group IA metal, a carbonate salt of a Group IA metal. The buffering agent can be, but is not limited to, an amino acid, an alkali metal salt of an ammo acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate coprecipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, 5 sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, trometamol, and mixtures thereof.

The buffering agent can be sodium bicarbonate, sodium carbonate, calcium carbonate, magnesium oxide, magnesium hydroxide, magnesium carbonate, aluminum hydroxide, and mixtures thereof.

The buffering agent can be used with the proton pump inhibitor in a ratio of at least 15 10:1; at least 12:1; at least 15:1; at least 20:1; at least 22:1; at least 25:1; at least 30:1; at least 35:1; and at least 40:1.

If the buffering agent is sodium bicarbonate, it can be used in the composition in amounts equivalent to about 0.1 mEq/mg proton pump inhibitor to about 5 mEq/mg proton pump inhibitor. If the buffering agent is a mixture of sodium bicarbonate and magnesium hydroxide, each buffering agent can be present in amount of about 0.1 mEq/mg proton pump inhibitor to about 5 mEq/mg proton pump inhibitor. Where the buffering agent is a mixture of sodium bicarbonate, calcium carbonate, and magnesium hydroxide, each buffering agent can be present in amounts of about 0.1 mEq/mg proton pump inhibitor to about 5 mEq/mg of the proton pump inhibitor.

The buffering agent can be used an amount of about 0.1 mEq/mg to about 5 mEq/mg of the proton pump inhibitor, or about 0.5 mEq/mg to about 3 mEq/mg of the proton pump inhibitor, or about 0.8 mEq/mg to about 2.5 mEq/mg of the proton pump inhibitor, or about 0.9 mEq/mg to about 2.0 mEq/mg of the proton pump inhibitor, or about 0.9 mEq/mg to about 1.8 mEq/mg of the proton pump inhibitor.

The composition can further contemplate having about 200 to 3000 mg of a buffering agent, or about 500 to about 2500 mg of a buffering agent, or about 1000 to about 2000 mg of a buffering agent, or about 1500 to about 2000 mg of a buffering agent.

Coatings, other than microencapsulation coatings for timed release purposes can be used such as enzymatic-controlled coatings, film coatings, sustained-release coatings, immediate-release coatings, and delayed-release coatings.

The composition can be provided in a dosage form as a powder, a tablet, a bite-disintegration tablet, a chewable tablet, a caplet, a capsule, an effervescent powder, a rapid disintegrating tablet or an aqueous suspension produced from powder.

The composition can be disposed on an adhesive patch for continuous transdermal absorption of the proton pump inhibitors, with or without the therapeutic amounts of the additional components mentioned so far.

Flavoring agents and other materials to enhance the therapeutic experience of the composition can be included. The flavoring agents can be, but are not limited to sweetening agents, diffusion facilitators, antioxidants, and carrier materials selected from binders, suspending agents, disintegration agents, filling agents, surfactants, solubilizers, stabilizers, wetting agents, diluents and antifoaming agents.

The invention further provides a process for the preparation of a pharmaceutical composition of the invention which comprises mixing at least one proton pump inhibitor and at least one insulin secretagogue, or a pharmaceutically acceptable salt, ester, or solvate thereof, as herein described, with a pharmaceutically acceptable adjuvant, diluent or carrier. The carrier(s) must be “acceptable” in the sense of being compatible with the other ingredients of the formulation and not deleterious to the recipient thereof.

The compositions may be administered topically (e.g., to the lung and/or airways or to the skin or other epithelial surface) in the form of solutions, suspensions, native or heptafluoroalkane aerosols and dry powder formulations; or systemically, e.g., by oral administration in the form of tablets, capsules, syrups, powders or granules, or by parenteral administration in the form of solutions or suspensions, or by subcutaneous administration or by vaginal or rectal administration in the form of suppositories, or transdermally.

Compositions of the present invention can be further combined with any other suitable additive or pharmaceutically acceptable carrier. Such additives include any of the substances already mentioned, as well as any of those used conventionally, such as those described in Remington: The Science and Practice of Pharmacy (Gennaro and Gennaro, eds, 20th edition, Lippincott Williams & Wilkins, 2000); Theory and Practice of Industrial Pharmacy (Lachman et al., eds., 3rd edition, Lippincott Williams & Wilkins, 1986); Encyclopedia of Pharmaceutical Technology (Swarbrick and Boylan, eds., 2nd edition, Marcel Dekker, 2002). These can be referred to herein as “pharmaceutically acceptable carriers” to indicate they are combined with the active drug and can be administered safely to a subject for therapeutic purposes.

The compositions of the present invention can be administered by any available and effective delivery system including, but not limited to, orally, bucally, within the ear or nasal passages, parenterally, by inhalation spray, by topical application, by injection, transdermally, vaginally or rectally (e.g., by the use of suppositories) in dosage unit formulations containing conventional nontoxic pharmaceutically acceptable carriers, adjuvants, and vehicles, as desired. Parenteral includes subcutaneous injections, intravenous, intramuscular, intrasternal injection, or infusion techniques.

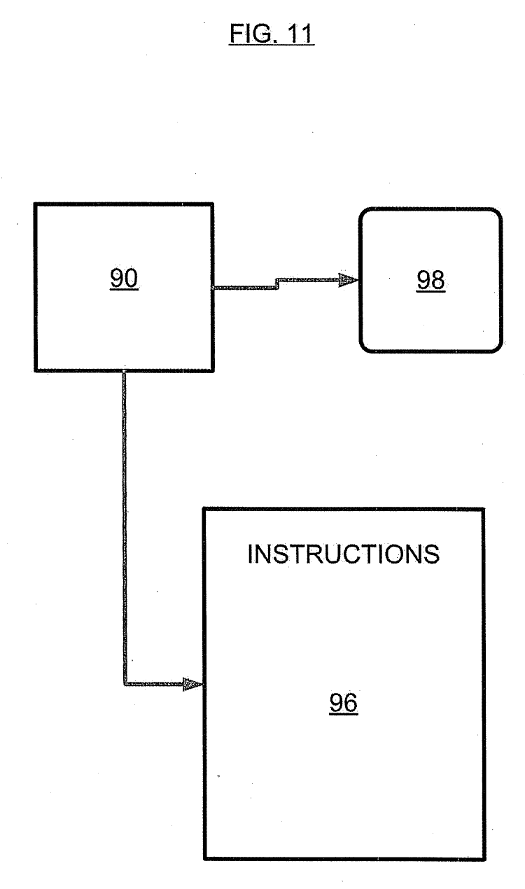

Transdermal administration, which is known to one skilled in the art, involves the delivery of pharmaceutical compounds via percutaneous passage of the compounds into the systemic circulation of the patient. Topical administration can also involve the use of transdermal administration such as transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, pastes, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400 which may function as solvent or absorption modifier. They can be mixed to form ointments with, for example, benzyl alcohol 2% (wt/wt) as preservative, white petrolatum, emulsifying wax, and Tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous cross linking agent impregnated with the composition and laminated to an impermeable backing FIG. 11 provides an illustration of how the composition of the present invention may be prepared as a kit [90] that contains a patch [98] having at least one proton pump inhibitor and at least one insulin secretagogue. In this example, the patch may contain sufficient amounts of at least one proton pump inhibitor and at least one insulin secretagogue to be used for one day or may be used for extended times such as at least one week, at least two weeks, at least three weeks, or at least one month.

Solid dosage forms for oral administration can include capsules, tablets, effervescent tablets, chewable tablets, pills, powders, sachets, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the present invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for vaginal or rectal administration of the compounds and compositions of the invention can be prepared by mixing the compounds or compositions with a suitable nonirritating excipient such as cocoa butter and polyethylene glycols which are solid at room temperature but liquid at body temperature, such that they will melt and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

The compositions of the invention can further include conventional excipients, i.e., pharmaceutically acceptable organic or inorganic carrier substances suitable for parenteral application which do not deleteriously react with the active compounds. Suitable pharmaceutically acceptable carriers include, for example, water, salt solutions, alcohol, vegetable oils, polyethylene glycols, gelatin, lactose, amylase, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances that increase the viscosity of the suspension and include, for example, sodium carboxymethyl cellulose, sorbitol and/or dextran. Optionally, the suspension may also contain stabilizers.

Solvents useful in the practice of this invention include pharmaceutically acceptable, water-miscible, non-aqueous solvents. In the context of this invention, these solvents should be taken to include solvents that are generally acceptable for pharmaceutical use, substantially water-miscible, and substantially non-aqueous. Preferably, these solvents are also non-phthalate plasticizer leaching solvents, so that, when used in medical equipment, they substantially do not leach phthalate plasticizers that may be present in the medical equipment. More preferably, the pharmaceutically-acceptable, water-miscible, non-aqueous solvents usable in the practice of this invention include, but are not limited to, N-methylpyrrolidone (NMP); propylene glycol; ethyl acetate; dimethyl sulfoxide; dimethyl acetamide; benzyl alcohol; 2-pyrrolidone; benzyl benzoate; C.sub.2-6 alkanols; 2-ethoxyethanol; alkyl esters such as 2-ethoxyethyl acetate, methyl acetate, ethyl acetate, ethylene glycol diethyl ether, or ethylene glycol dimethyl ether; (S)-(−)-ethyl lactate; acetone; glycerol; alkyl ketones such as methyl ethyl ketone or dimethyl sulfone; tetrahydrofuran; cyclic alkyl amides such as caprolactam; decylmethylsulfoxide; oleic acid; aromatic amines such as N,N-diethyl-m-toluamide; or 1-dodecylazacycloheptan-2-one.

The compositions of this invention can further include solubilizers. Solubilization is a phenomenon that enables the formation of a solution. It is related to the presence of amphiphiles, that is, those molecules that have the dual properties of being both polar and non-polar in the solution that have the ability to increase the solubility of materials that are normally insoluble or only slightly soluble, in the dispersion medium. Solubilizers often have surfactant properties. Their function may be to enhance the solubility of a solute in a solution, rather than acting as a solvent, although in exceptional circumstances, a single compound may have both solubilizing and solvent characteristics. Solubilizers useful in the practice of this invention include, but are not limited to, triacetin, polyethylene glycols (such as, for example, PEG 300, PEG 400, or their blend with 3350, and the like), polysorbates (such as, for example, Polysorbate 20, Polysorbate 40, Polysorbate 60, Polysorbate 65, Polysorbate 80, and the like), poloxamers (such as, for example, Poloxamer 124, Poloxamer 188, Poloxamer 237, Poloxamer 338, Poloxamer 407, and the like), polyoxyethylene ethers (such as, for example, Polyoxyl 2 cetyl ether, Polyoxyl 10 cetyl ether, and Polyoxyl 20 cetyl ether, Polyoxyl 4 lauryl ether, Polyoxyl 23 lauryl ether, Polyoxyl 2 oleyl ether, Polyoxyl 10 oleyl ether, Polyoxyl 20 oleyl ether, Polyoxyl 2 stearyl ether, Polyoxyl 10 stearyl ether, Polyoxyl 20 stearyl ether, Polyoxyl 100 stearyl ether, and the like), polyoxylstearates (such as, for example, Polyoxyl 30 stearate, Polyoxyl 40 stearate, Polyoxyl 50 stearate, Polyoxyl 100 stearate, and the like), polyethoxylated stearates (such as, for example, polyethoxylated 12-hydroxy stearate, and the like), and Tributyrin.

Other materials that may be added to the compositions of the present invention include cyclodextrins, and cyclodextrin analogs and derivatives, and other soluble excipients that could enhance the stability of the inventive composition, maintain the product in solution, or prevent side effects associated with the administration of the inventive composition. Cyclodextrins may be available as ENCAPSIN™ from Janssen Pharmaceuticals.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and the like.

Various delivery systems are known and can be used to administer the compounds or compositions of the present invention, including, for example, encapsulation in liposomes, microbubbles, emulsions, microparticles, microcapsules, nanoparticles, and the like. The required dosage can be administered as a single unit or in a sustained release form.

The bioavailabilty of the compositions can be enhanced by micronization of the formulations using conventional techniques such as grinding, milling, spray drying and the like in the presence of suitable excipients or agents such as phospholipids or surfactants.

Sustained release dosage forms of the invention may comprise microparticles and/or nanoparticles having a therapeutic agent dispersed therein or may comprise the therapeutic agent in pure, preferably crystalline, solid form. For sustained release administration, microparticle dosage forms comprising pure, preferably crystalline, therapeutic agents are preferred. The therapeutic dosage forms of this aspect of the present invention may be of any configuration suitable for sustained release. Preferred sustained release therapeutic dosage forms exhibit one or more of the following characteristics: microparticles (e.g., from about 0.5 micrometers to about 100 micrometers in diameter, preferably about 0.5 to about 2 micrometers; or from about 0.01 micrometers to about 200 micrometers in diameter, preferably from about 0.5 to about 50 micrometers, and more preferably from about 2 to about 15 micrometers) or nanoparticles (e.g., from about 1.0 nanometer to about 1000 nanometers in diameter, preferably about 50 to about 250 nanometers; or from about 0.01 nanometer to about 1000 nanometers in diameter, preferably from about 50 to about 200 nanometers), free flowing powder structure; biodegradable structure designed to biodegrade over a period of time between from about 0.5 to about 180 days, preferably from about 1 to 3 to about 150 days, more preferably from about 3 to about 180 days, and most preferably from about 10 to about 21 days; or non-biodegradable structure to allow the therapeutic agent diffusion to occur over a time period of between from about 0.5 to about 180 days, more preferably from about 30 to about 120 days; or from about 3 to about 180 days, more preferably from about 10 to about 21 days; biocompatible with target tissue and the local physiological environment into which the dosage form to be administered, including yielding biocompatible biodegradation products; facilitate a stable and reproducible dispersion of therapeutic agent therein, preferably to form a therapeutic agent-polymer matrix, with active therapeutic agent release occurring by one or both of the following routes: (1) diffusion of the therapeutic agent through the dosage form (when the therapeutic agent is soluble in the shaped polymer or polymer mixture defining the dimensions of the dosage form); or (2) release of the therapeutic agent as the dosage form biodegrades; and/or for targeted dosage forms, capability to have, preferably, from about 1 to about 10,000 binding protein/peptide to dosage form bonds and more preferably, a maximum of about 1 binding peptide to dosage form bond per 150 square angstroms of particle surface area. The total number of binding protein/peptide to dosage form bonds depends upon the particle size used. The binding proteins or peptides are capable of coupling to the particles of the therapeutic dosage form through covalent ligand sandwich or non-covalent modalities as set forth herein.

Nanoparticle sustained release therapeutic dosage forms are preferably biodegradable and, optionally, bind to the vascular smooth muscle cells and enter those cells, primarily by endocytosis. The biodegradation of the nanoparticles occurs over time (e.g., 30 to 120 days; or to 21 days) in prelysosomic vesicles and lysosomes. Preferred larger microparticle therapeutic dosage forms of the present invention release the therapeutic agents for subsequent target cell uptake with only a few of the smaller microparticles entering the cell by phagocytosis. A practitioner in the art will appreciate that the precise mechanism by which a target cell assimilates and metabolizes a dosage form of the present invention depends on the morphology, physiology and metabolic processes of those cells. The size of the particle sustained release therapeutic dosage forms is also important with respect to the mode of cellular assimilation. For example, the smaller nanoparticles can flow with the interstitial fluid between cells and penetrate the infused tissue. The larger microparticles tend to be more easily trapped interstitially in the infused primary tissue, and thus are useful to deliver anti-proliferative therapeutic agents.

Preferred sustained release dosage forms of the present invention comprise biodegradable microparticles or nanoparticles. More preferably, biodegradable microparticles or nanoparticles are formed of a polymer containing matrix that biodegrades by random, nonenzymatic, hydrolytic scissioning to release therapeutic agent, thereby forming pores within the particulate structure.

The compositions of the present invention can be formulated as pharmaceutically acceptable esters or salts. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitrous (nitrite salt), nitric (nitrate salt), carbonic, sulfuric, phosphoric acid, and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, parahydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, .beta.-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

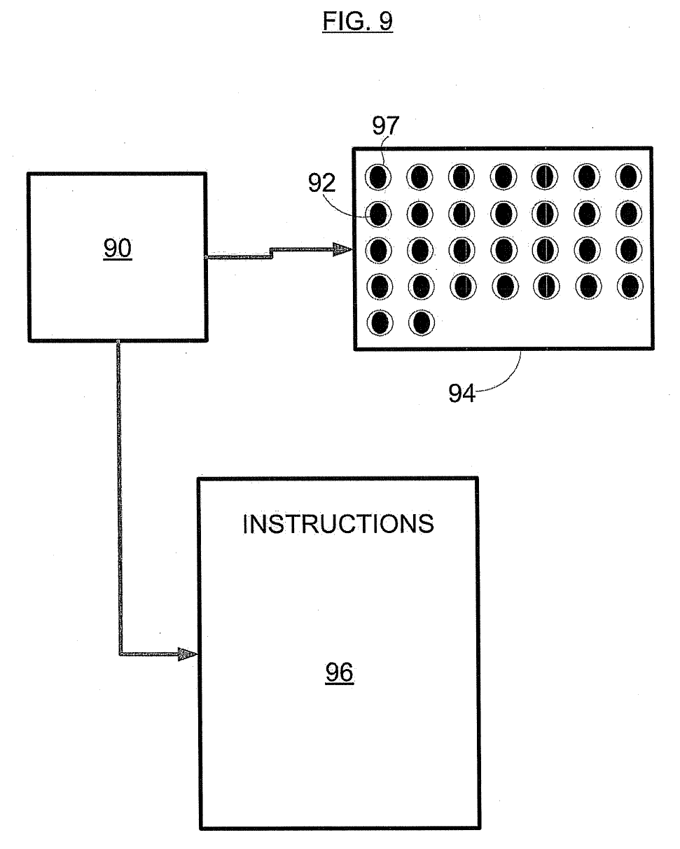

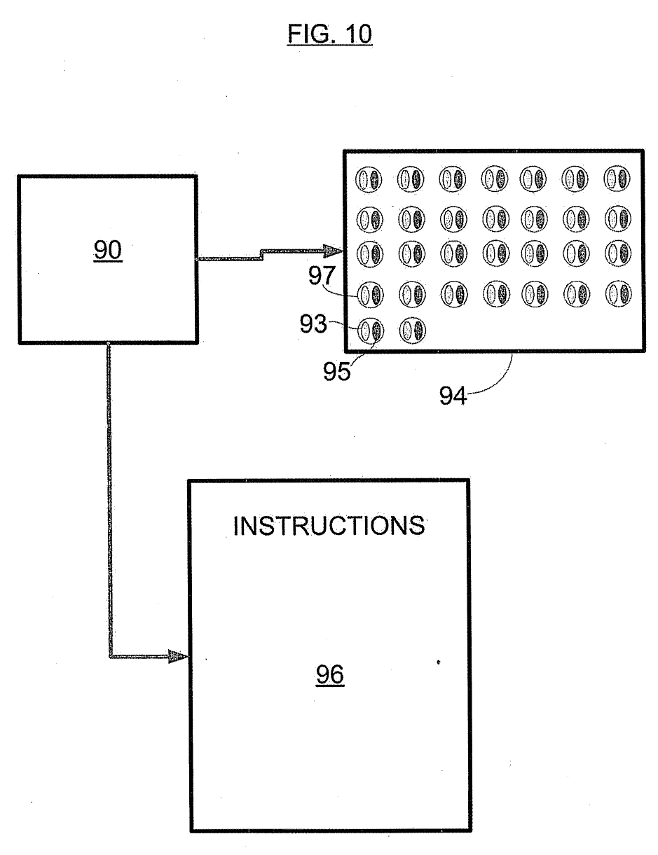

In one embodiment, the invention provides kits comprising the therapeutic combination of the invention, or the pharmaceutical composition of the invention. The kit can comprise at least one package, e.g., a blister pack, containing any one or more of a therapeutic combination of any of the invention, or the pharmaceutical composition of the invention. The kits of the present invention can optionally contain instructions associated with the dosage units of the kits. Such instructions can be in a form prescribed by a governmental agency regulating the manufacture, use or sale of pharmaceuticals or biological products, which notice reflects approval by the agency of the manufacture, use or sale for human administration to treat a condition or disorder. The instructions can be in any form which conveys information on the use of the dosage units in the kit according to the methods of the invention. For example, the instructions can be in the form of printed matter, or in the form of a pre-recorded media device. For example, in some embodiments of the present invention, the kit contains at least 7, at least 10, at least 14, at least 21, or at least 30 tablets for oral administration, each tablet containing a combination of at least one proton pump inhibitor and at least one insulin secretagogue and intended for ingestion on successive days. FIG. 9 provides an illustration of a kit [90] that contains 30 pills in a blister pack [94] and instructions on how to use the pills [96]. In this example, each well [97] contains a pill containing [92] at least one proton pump inhibitor and at least one insulin secretagogue. FIG. 10 provides an illustration of a kit [90] that contains 60 wells—30 pills containing at least one proton pump inhibitor and 30 pills containing at least one insulin secretagogue. In this example, the pills would be arranged in pairs in each well [97] so that one pill containing at least one proton pump inhibitor [93] and one pill containing at least one insulin secretagogue [96] would be placed in each well [97] of the blister pack [94]. The kits of the present invention can optionally contain instructions associated with the dosage units of the kits. Such instructions can be in a form prescribed by a governmental agency regulating the manufacture, use or sale of pharmaceuticals or biological products, which notice reflects approval by the agency of the manufacture, use or sale for human administration to treat a condition or disorder. The instructions can be in any form which conveys information on the use of the dosage units in the kit according to the methods of the invention. For example, the instructions can be in the form of printed matter, or in the form of a pre-recorded media device. Item [96] is an illustration of instructions for the kits of FIG. 9, FIG. 10, and FIG. 11.

“Printed matter” can be, for example, one of a book, booklet, brochure or leaflet. The printed matter can describe the use of the dosage units of the kit according to the regimens of the present invention. Possible formats include, but are not limited to, a bullet point list, a list of frequently asked questions (FAQ) or a chart. Additionally, the information to be imparted can be illustrated in non-textual terms using pictures, graphics or other symbols.

“Pre-recorded media device” can be, for example, a visual media device, such as a videotape cassette, a DVD (digital video disk), filmstrip, 35 mm movie or any other visual media device. Alternately, pre-recorded media device can be an interactive software application, such as a CD-ROM (compact disk-read only memory) or floppy disk. Alternately, pre-recorded media device can be, for example, an audio media device, such as a record, audiocassette or audio compact disk. The information contained on the pre-recorded media device can describe the proper use of the dosage units in the kit for the treatment of one or more of the conditions or disorders as described herein.

In addition to instructions, the kit can optionally contain a planner A “planner” can be, for example, a weekly, a monthly, a multi-monthly, a yearly, or a multi-yearly planner. The planner can be used as a diary to monitor dosage amounts, to keep track of dosages administered, or to prepare for future events wherein taking the dosages of the kit can be difficult. Alternately, the planner can be a calendar which will provide a means to monitor when a dosage has been taken and when it has not been taken. This type of planner will be particularly useful for patients having unusual schedules for administering medication to themselves. One skilled in the art will appreciate the variety of planning tools that would be appropriate for use with the present invention.

The kit can also include a container for storing the other components of the kit. The container can be, for example, a bag, box, envelope or any other container that would be suitable for use in the present invention. The container can be large enough to accommodate each component and/or any administrative devices that can be necessary for use of the dosage units of the kit according to the methods of the present invention. However, in some cases, it can be desirable to have a smaller container which can be hidden in a pocketbook, briefcase or pocket.

While individual needs may vary, determination of optimal ranges for effective amounts of the compositions is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

### EXAMPLES

The following three examples further illustrate the composition.

### Example 1

For a population of 347 Type II diabetic patients with recent HgbA1c measurements of an average 7.53% with a standard deviation of +/−1.77%. All were treated using insulin injections and/or a variety of oral agents, such as the above noted metformin, and other anti-diabetic medicines. The patients were treated for differing periods of time ranging from 3 months to 30 years.

It was found that the population of 347 could be subdivided into two groups, 282 treated for diabetes but not taking a proton pump inhibitor (PPI) and 65 taking a PPI.

The two groups were compared using a two-Sample T test that is a classical statistical test. In comparison, it was found that the 282 people treated for diabetes but not taking a PPI, had a test results for HgbA1c averaging 7.64%, with a standard deviation of 1.85%. In contrast, the 65 taking a PPI, had an average HgbA1c test result of 7.03%, with a standard deviation of 1.26%. The difference between the two groups was 0.61%, which is a significant difference with a p-value of 0.002. This results indicated that there was only 1 chance in 500 that this lowering of the HgbA1c was a coincidence. This population study indicated that the PPI effectively increases either insulin production or increases beta cell mass capable of insulin production in the pancreas, lowering average blood sugar for this population.

### Example 2

64 total patients, of which 53 patients that were treated using only Metformin for the treatment of diabetes and 11 took Metformin and a proton pump inhibitor.

For the Metformin only patients, the HgbA1c was and average of 7.27% with a standard deviation of 1.75%. In contrast, the 11 patients the average HgbA1c were 6.55% with a standard deviation of only 0.88%. The difference between the two groups was 0.72% which is a significant difference with a p-value of 0.05. The patients were treated for differing periods of time ranging from 3 months or more.

### Example 3

In this population, 79 patients were dosed with a sulfonylurea, either alone or in combination with metformin and/or pioglitazone or rosiglitazone. These 79 patients were not provided with insulin or sitagliptin.

The 79 patients had an average HgbA1c of 7.86%, with a standard deviation of 1.92%.

10 patients were identical to the 79 except they additionally were provided dosage amounts of a proton pump inhibitor. For this group of 10, the average HgbA1c was 6.52%, with a standard deviation of only 0.69%. The difference between the two groups was 1.34%, which is a significant difference in p-value of <0.001. This showed that it was less than 1 and 1000 that there could have been a coincidence in the test results of the HgbA1c. The patients were treated for differing periods of time ranging from 3 months or more.

### Example 4

Seven Type II diabetic patients were administered omeprazole for 24 weeks and data was obtained from fasting and following a 2 hour glucose tolerance test at 8 week intervals and compared to baseline values. Statistical comparisons and analysis were performed with a commercial program, Analyse-it, version 2.20.

Subjects: Five males, 2 females, age range 42-68 years, mean age 54+10 years. BMI's ranged from 31 to 49, mean 38+6. All subjects had co-morbid hypertension, four had co-morbid dyslipidemia and two had co-morbid hypothyroidism. All were stable on oral diabetes medications with 7/7 on metformin, 6/7 on pioglitazone (Actos), 4/7 on a DPP-4 inhibitor, sitagliptan (Januvia) and 4/7 on a Sulfonylurea. All subjects received a physical exam, review of systems and a baseline EKG prior to initiating the PPI trial. Subjects were then seen and evaluated at monthly intervals interviewed for adverse events and monitored for medication compliance and maintenance of daily blood glucose logs.

Design: All subjects continued on their respective oral antidiabetic medications throughout the study. Baseline data was obtained in a fasting state after an overnight fast. Subjects were then administered 75 g of glucose and blood samples obtained at 1 and 2 hours. Following collection of baseline data, subjects were administered Omeprazole Delayed-Release Capsules USP, (Apotex Inc., Toronto, Canada M9L 179), 40 mg orally twice daily and blood samples again obtained after 8, and 16 weeks. Medications were not taken on the mornings of testing. Following the 16 week sample collection, subjects were increased to 80 mg Omeprazole twice daily and blood samples again obtained at 24 weeks.

Measurements: Helicobacter Pylori IgG was obtained on each subject as a baseline as were Hemoglobin A1c and fasting Gastrin. Each 2 hour glucose tolerance test provided fasting glucose, insulin and c-peptide as well as 1 hour and 2 hour glucose and stimulated insulin and c-peptide. All measurements were obtained through a commercial laboratory using standardized analytical methods, Quest Diagnostics Laboratory, 5850 Rogerdale Road, Houston, Tex. 77072.

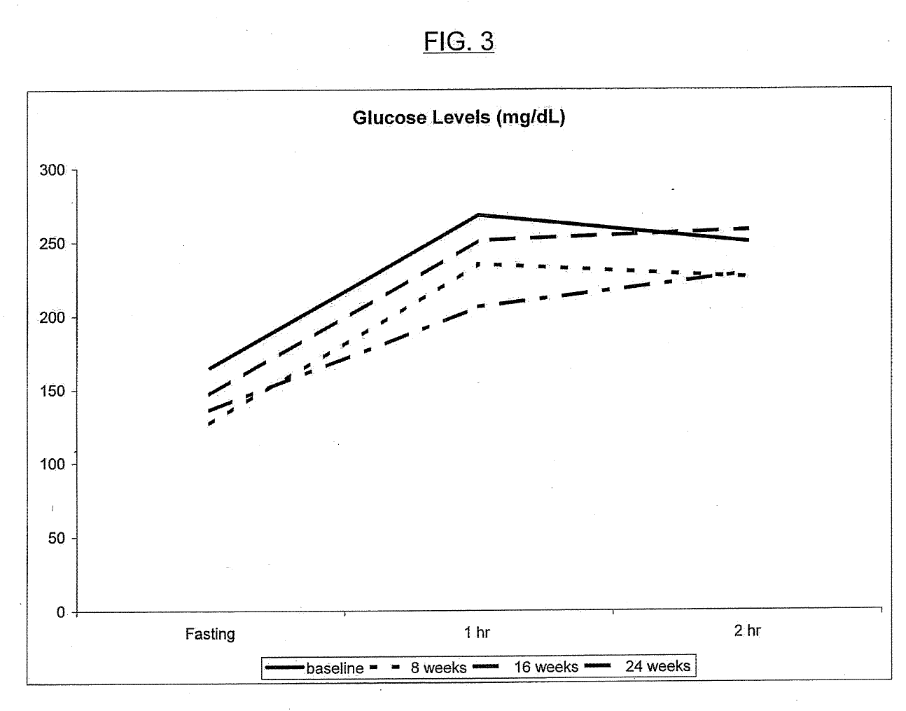

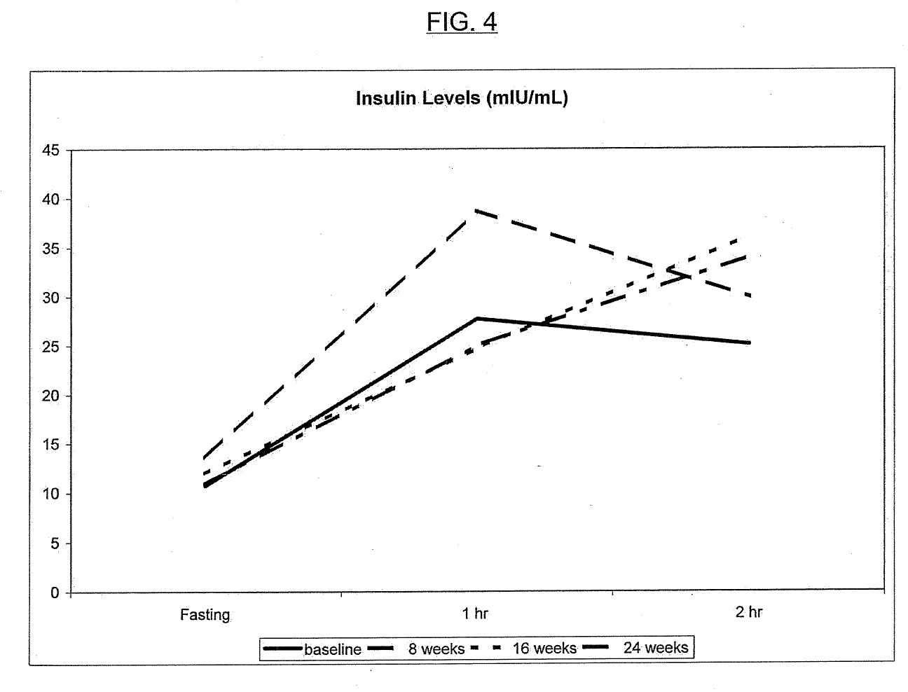

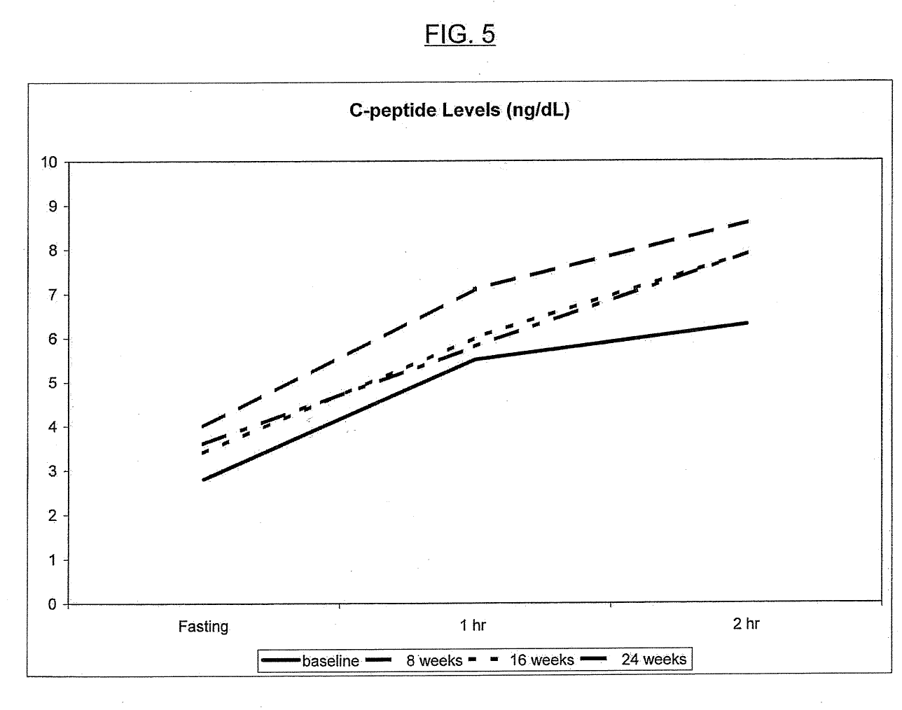

Results: Omeprazole was well tolerated at all doses with no reports of adverse events. Table 1 summarizes the observed effects of Omeprazole on indices of diabetes severity or control after the 2 hour glucose tolerance test. Fasting blood glucose was reduced at both 8 and 16 weeks as was the maximum glucose excursion at both 1 and 2 hours with statistically significant reductions at 16 weeks (FIG. 3). Baseline fasting records were obtained in all but one subjects. Stimulated insulin secretion at both 1 and 2 hours was increased after PPI administration although fasting insulin levels were unchanged (FIG. 4). Similarly, 1 and 2 hour c-peptide secretion was increased after PPI administration (FIG. 5).

TABLE 1
Effects of PPI Administration on Diabetes Indices
following a 2 Hour Glucose Tolerance Test.

| Test | Glucose | 1 | 2 | Insulin | 1 | 2 | C-Peptide | 1 | 2 |
|---|---|---|---|---|---|---|---|---|---|
| Period | Fasting | hour | hours | Fasting | hour | hours | Fasting | hour | hours |
| Baseline | 165 | 268 | 250 | 10.7 | 27.7 | 25.1 | 2.8 | 5.5 | 6.3 |
| 8 weeks | 127 | 235# | 226 | 13.6 | 38.7 | 29.9 | 4.0 | 7.1 | 8.6*** |
| PPI | | | | | | | | | |
| 16 | 136 | 206* | 229** | 12 | 24.7 | 36.1 | 3.4 | 6.0 | 7.9# |
| weeks | | | | | | | | | |
| PPI | | | | | | | | | |
| 24 | 147 | 251 | 258 | 10.9 | 25 | 34 | 3.6 | 5.8 | 7.9+ |
| weeks | | | | | | | | | |
| PPI | | | | | | | | | |

#P = 0.06,
*P = 0.04,
**P = 0.018,
***P < 0.001,
+P = 0.048

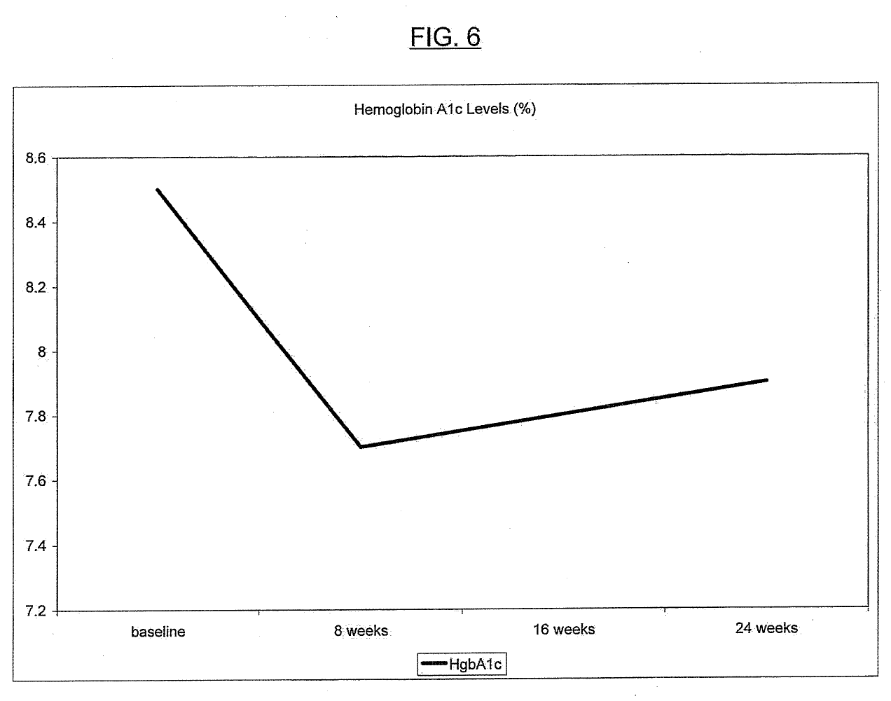

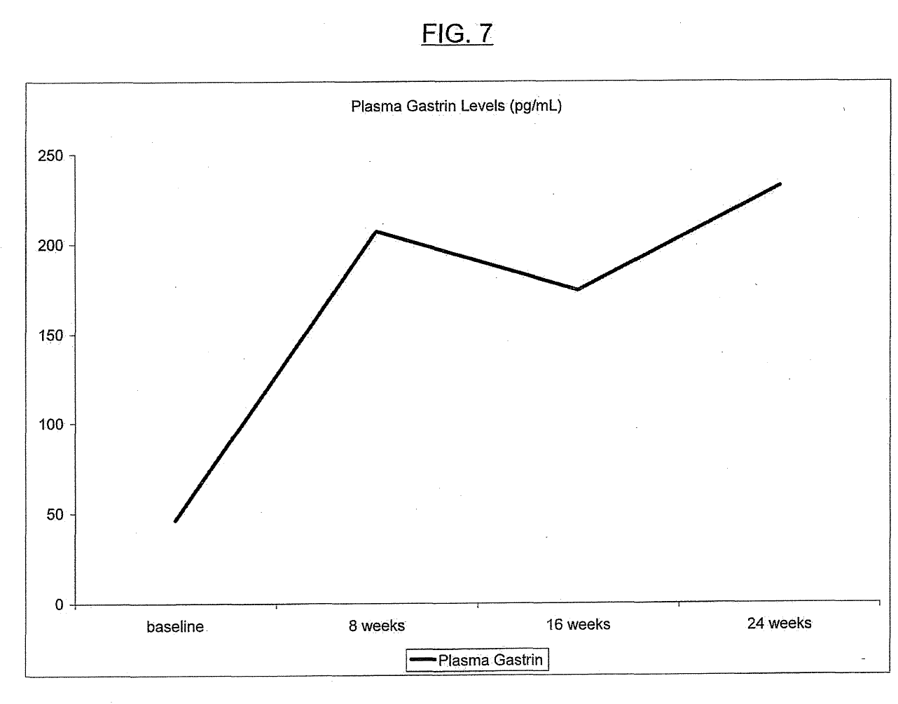

Table 2 summarizes the effects of PPI administration on plasma Gastrin, Hemoglobin A1c and % Beta Cell area (calculated using the 2 hour glucose tolerance value by the method of Meier et al, Diabetes, vol 58, 1595-1603, 2009). As expected, plasma gastrin was markedly elevated by long term PPI administration, although serum levels gradually reduced over time (FIG. 7). Hemoglobin A1c, a measure of average blood glucose, was reduced by approximately 0.9% in this cohort, consistent with improved diabetes control (FIG. 6). Finally, the calculated Beta cell mass increased across the treatment period by approximately 25%.

TABLE 2
Effects of PPI administration on Hemoglobin A1c %, Beta cell area, and
plasma gastrin.

| | | Beta | Plasma Gastrin, |
|---|---|---|---|
| Test Period | Hemoglobin A1 c, % | Cell Area, % | pg/ml |
| Baseline | 8.5 | 0.41 | 46 |
| 8 weeks PPI | 7.7 | 0.46 | 207 |
| 16 weeks PPI | 7.8 | 0.51 | 174 |
| 24 weeks PPI | 7.9 | 0.40 | 232 |

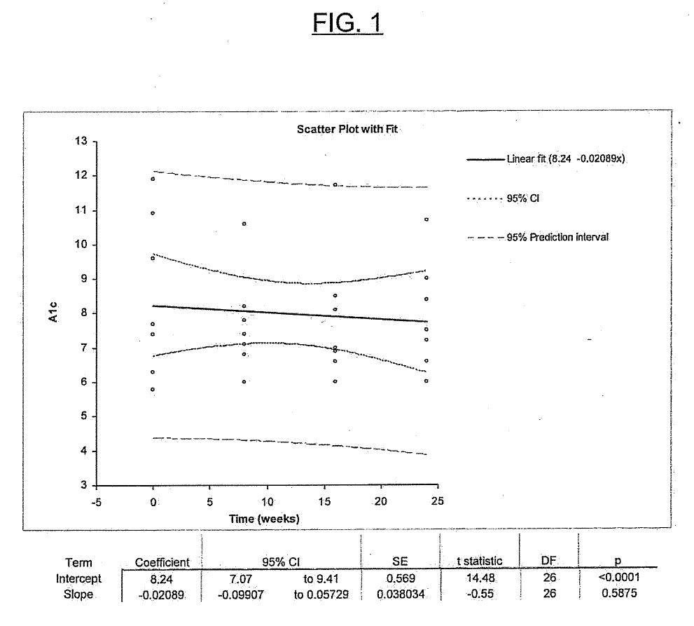

FIG. 1 shows a scatter plot of all hemoglobin A1c data obtained for seven type II diabetics across a 24 week trial of PPI administration. Best fit for the group was linear showing a slope of −0.021% (HgbA1c) per week. This demonstrates that in a group of diabetics, on a variety of oral medications that concurrent PPI administration results in a gradual reduction of hemoglobin A1c, consistent with improving diabetes control.

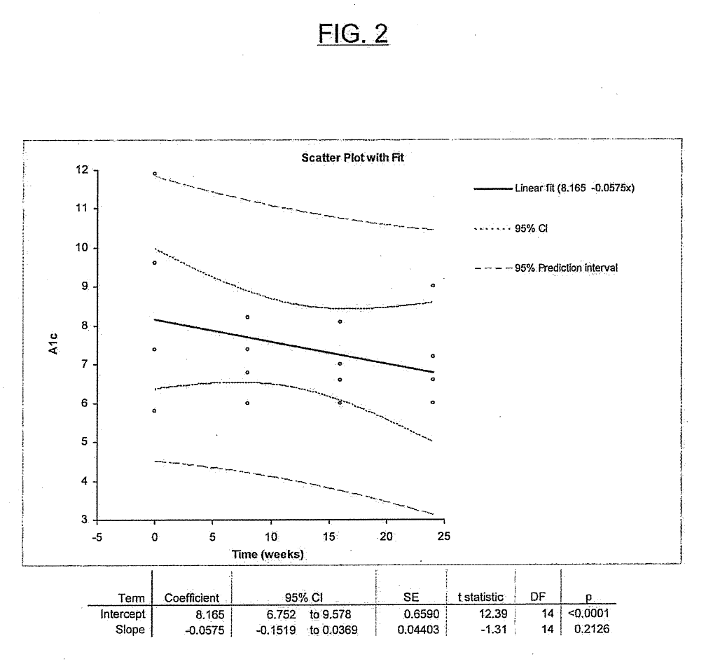

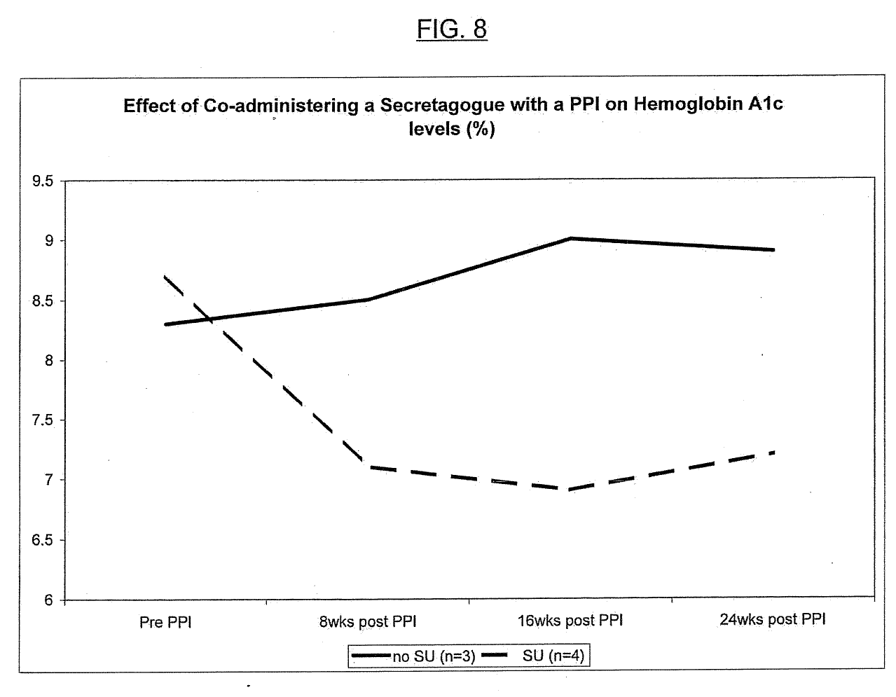

FIG. 2 shows a scatter plot of the hemoglobin A1c values for four type II diabetics during the 24 week trial of PPI administration, concurrently taking a sulfonylurea (secretagogue). Again the best fit was linear with a slope of −0.058% (HgbA1c) per week. This can also be seen in FIG. 8. This reduction represents a 2.5 fold relative rate of decrease in hemoglobin A1c relative to the group as a whole, consistent with enhanced effect of the secretagogue/PPI combination.

While these embodiments have been described with emphasis on the embodiments, it should be understood that within the scope of the appended claims, the embodiments might be practiced other than as specifically described herein.

Although the invention has been shown in only a few of its forms, it should be apparent to those skilled in the art that it is not so limited but susceptible to various changes without departing from the scope of the invention. Accordingly, it is intended to embrace all such alternatives, modifications, and variations as fall within the spirit and broad scope of the appended claims.

Those skilled in the art will recognize that many changes and modifications may be made to the method of practicing the invention without departing the scope and spirit of the invention. In the drawings and specification, there have been disclosed embodiments of the invention and, although specific terms are employed, they are used in a generic and descriptive sense only and not for the purpose of limitation, the scope of the invention being set forth in the following claims. The invention has been described in considerable detail with specific reference to these illustrated embodiments. It will be apparent, however, that various modifications and changes can be made within the spirit and scope of the invention as described in the foregoing specification. Furthermore, language referring to order, such as first and second, should be understood in an exemplary sense and not in a limiting sense. For example, it may be recognized by those skilled in the art that certain steps can be combined into a single step.

What is claimed is:

1-64. (canceled)

65. A composition to treat diabetes in a mammal having diabetes, the composition comprising: a therapeutically effective amount of at least one proton pump inhibitor and a therapeutically effective amount of at least one insulin secretagogue.

66. The composition according to claim 65 in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

67. The composition of claim 65, wherein the amount of the at least one proton pump inhibitor and the amount of the at least one insulin secretagogue will reduce hemoglobin A1c in the mammal by at least 1.5%.

68. The composition of claim 67, wherein the at least one proton pump inhibitor and the at least one insulin secretagogue are administered to the mammal daily for a period of at least 6 weeks and up to 24 weeks.

69. The composition of claim 67, wherein the at least one proton pump inhibitor and the at least one insulin secretagogue are administered to the mammal daily for a period of at least 6 weeks.

70. The composition of claim 67, wherein the at least one proton pump inhibitor and the at least one insulin secretagogue are administered to the mammal daily for a period of at least 24 weeks.

71. The composition of claim 65, wherein the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, pariprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof.

72. The composition of claim 65, wherein the at least one proton pump inhibitor is given to the mammal in a daily dose of at least 10 mg and the at least one insulin secretagogue is give to the mammal in a daily dose of at least 0.1 mg.

73. The composition of claim 65, wherein the at least one proton pump inhibitor is given to the mammal in a daily range of at least 5 mg and at most 1,000 mg and the at least one insulin secretagogue is give to the mammal in a daily range of at least 0.1 mg and at most 500 mg.

74. A composition to treat diabetes in a mammal having diabetes, the composition comprising: a therapeutically effective amount of at least one proton pump inhibitor and a therapeutically effective amount of at least one insulin secretagogue so that the at least one proton pump inhibitor and the at least one insulin secretagogue when administered to the mammal cause an increase of the mammal's gastrin concentration levels compared to the mammal's baseline, the gastrin concentration levels increase thereof leading to a reduction of the mammal's hemoglobin A1C.

75. The composition according to claim 74 in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.

76. The composition of claim 74, wherein the amount of the at least one proton pump inhibitor and the amount of the at least one insulin secretagogue will reduce hemoglobin Ale in the mammal by at least 1.5%.

77. The composition of claim 76, wherein the at least one proton pump inhibitor and the at least one insulin secretagogue are administered to the mammal daily for a period of at least 6 weeks and up to 24 weeks.

78. The composition of claim 76, wherein the at least one proton pump inhibitor and the at least one insulin secretagogue are administered to the mammal daily for a period of at least 6 weeks.

79. The composition of claim 76, wherein the at least one proton pump inhibitor and the at least one insulin secretagogue are administered to the mammal daily for a period of at least 24 weeks.

80. The composition of claim 74, wherein the at least one proton pump inhibitor is selected from the group consisting essentially of omeprazole, lansoprazole, esomeprazole, rabeprazole, pantoprazole, pariprazole, tenatoprazole, leminoprazole, hydroxyomeprazole, dontoprazole, habeprazole, periprazole, or a free base, free acid, salt, hydrate, ester, amide, enantiomer, isomer, tautomer, polymorph, or prodrug thereof and the at least one insulin secretagogue is selected from the group consisting essentially of acetohexamide, chlorpropamide, tolbutamide, tolazamide, glipizide, gliclazide, glibenclamide, gliquidone, glyclopyramide, glimepiride, neteglinide, repaglinide, or a free base, free acid, salt, hydrate, ester, amide, enantiomer, isomer, tautomer, polymorph, or prodrug, and combinations thereof.

81. The composition of claim 74, wherein the at least one proton pump inhibitor is given to the mammal in a daily dose of at least 10 mg and the at least one insulin secretagogue is give to the mammal in a daily dose of at least 0.1 mg.

82. The composition of claim 74, wherein the at least one proton pump inhibitor is given to the mammal in a daily range of at least 5 mg and at most 1,000 mg and the at least one insulin secretagogue is give to the mammal in a daily range of at least 0.1 mg and at most 500 mg.

83. A composition for reducing hemoglobin A1C in a mammal, the composition comprising: a therapeutically effective amount of at least one proton pump inhibitor and a therapeutically effective amount of at least one insulin secretagogue.

84. The composition according to claim 83 in the form of a suspension, emulsion, aerosol, ointment, cream, gel, paste, suppository, stick, powder, topical powder, granular material, tablet, transdermal patch, pastille, sugar-coated pill, film-coated tablet, hard gelatin capsule, soft gelatin capsule, extrudate, microcapsule or a microsphere.
